(19) United States
(12) Patent Application Publication
(10) Pub. No.: US 2024/0336861 A1
(43) Pub. Date: Oct. 10, 2024

(54) MULTIPLE PRODUCT PATHWAY FROM RENEWABLE OILS TO PETROLEUM ALTERNATIVES AND LUBRICANTS COMPRISING SAME

(71) Applicant: Evolve Lubricants, Inc., Reno, NV (US)

(72) Inventors: Richard D. Lee, Reno, NV (US); Thomas L. Kirkham, Jr., Reno, NV (US); Erik Anderson, Reno, NV (US); Michael Doyle, Reno, NV (US)

(73) Assignee: Evolve Lubricants, Inc., Reno, NV (US)

(21) Appl. No.: 18/290,649

(22) PCT Filed: Jul. 21, 2022
(86) PCT No.: PCT/US2022/073996

§ 371 Date: Jan. 19, 2024

Related U.S. Application Data
(60) Provisional application No. 63/224,245, filed on Jul. 21, 2021.
(60) Provisional application No. 63/232,566, filed on Aug. 12, 2021.

(51) Int. Cl. C10M 105/04; C07C 1/213; C07C 11/02; C10L 1/06; C10L 1/08; C10N 20/00; C10N 20/02; C10N 30/00; C10N 30/02; C10N 30/04; C11C 1/02; C11C 1/08

(52) U.S. Cl. CPC C10M 105/04; C07C 1/213; C07C 11/02; C10L 1/06; C10L 1/08; C11C 1/025; C11C 1/08; C10L 2200/0476; C10M 2203/0206; C10N 2020/02; C10N 2020/071; C10N 2030/02; C10N 2030/04; C10N 2030/40

(57) ABSTRACT

A method for production of renewable hydrocarbons, including alpha olefins, renewable diesel, synthetic gasoline, and acyl-glycerides, from renewable oils is described herein. Also included is a method for production comprising (a) blending a specific renewable oil mixture with the proper free fatty acid character; (b) acid hydrolysis of the free fatty acids and subsequent purification of the unsaturated and saturated chains; (c) converting the saturated portion into renewable diesel; and (d) reacting the unsaturated free fatty acids via ethenolysis to form alpha olefins, then converting the remaining free fatty acids into either synthetic gasoline or into an acyl-glycerol via glycerolysis.

### CROSS-REFERENCE TO RELATED APPLICATIONS

This application claims the benefit of priority to U.S. Provisional Patent Application Nos. 63/224,245, filed Jul. 21, 2021, and 63/232,566, filed Aug. 12, 2021, all of which are incorporated by reference herein in their entireties.

### FIELD OF THE INVENTION

Aspects of present disclosure generally relate to a single method of production for multiple unique renewable hydrocarbons products for various industries, including processes for the formation of long and short-chain alpha olefins, saturated hydrocarbons, and acyl-glycerides.

### BACKGROUND

Regarding the global economy, it is recognized that increasing energy demands coupled with the depletion of non-renewable resources requires a significant paradigm shift to the way energy and materials are made. Concerns for environmental issues are forcing industries to look for alternative resources more now than in previous years. Hence, substituting biogenic, organic material for petroleum-based materials has been the focus of an increasing number of research institutes and companies in the last decades. Organic components such as carbohydrates, glycerol, free fatty acids, lignocellulose, and amino acids have been researched as alternative precursors for many fuel types and chemicals currently produced from petroleum sources. There is currently a complete lack of higher performing bio-lubricant and carbon negative options, and therefore industries are obliged to utilize petroleum lubricants due to the absence of non-petroleum products capable of meeting manufacturer performance specifications.

### SUMMARY

In aspects, the disclosure provides a method for preparing a base oil from a renewable oil comprising triglycerides comprising:
a) acidifying the renewable oil to produce a mixture comprising;
   i) free fatty acid mixture comprising;
      saturated free fatty acids and unsaturated free fatty acids, and
   ii) glycerin;
b) isolating the glycerin from the mixture of fatty acids;
c) separating the saturated free fatty acids from the unsaturated free fatty acids;
d) subjecting the unsaturated free fatty acids to ethenolysis to prepare a mixture comprising;
   i) alpha olefins; and
   ii) short-chain unsaturated fatty acids, optionally C6-C12 or C8-C12 short chain unsaturated fatty acids; and
e) combining the glycerin from a) with at least a portion of the short chain unsaturated fatty acid of d) to produce a mixture and subjecting the mixture to glycerolysis.

In some embodiments, the method further includes decarboxylating at least a portion of the short chain unsaturated fatty acids of d) to produce saturated hydrocarbons, optionally wherein the decarboxylation comprises a catalyst or gas phase decarboxylation, optionally a Ni/C catalyst or an oxidative metal catalyst (e.g. silver (II)). In some embodiments, in a), the renewable oil is purified prior to acidification. In some embodiments, purifying the renewable oils comprises clarification, degumming, bleaching, and/or filtering. In some embodiments, in a), acidifying the renewable oil comprises contacting the renewable oil with an aqueous acid and an organic solvent to provide an organic fraction and an aqueous fraction, wherein the organic fraction comprises the free fatty acids mixture and the aqueous fraction comprises the glycerin. In some embodiments, in a), acidifying the renewable oil comprises heating a mixture of the renewable oil and water at a suitable pressure. In some embodiments, the mixture of the renewable oil and water at a suitable pressure comprising one or more of the following: a ratio of renewable oil to water ranges from about 5:1 to about 1:5, about 4:1 to about 1:4, about 3:1 to about 1:3, or about 2:1 to about 1:2 based on the total weight of the renewable oil and water; the mixture is heated to a temperature ranging from about 100° C. to about 350° C., about 200° C. to about 300° C., or about 250° C. to about 275° C.; and the pressure ranges from about 500 psi to about 1000 psi, about 700 psi to about 900 psi, or about 800 psi to about 900 psi. In some embodiments, the acidifying is repeated more than once. In some embodiments, the acid comprises at least one of H₂SO₄, HCl, and H₃PO₄. In some embodiments, the organic fraction comprises 90 wt. % to about 100 wt % free fatty acids and about 0 wt. % to about 10 wt. % glycerin. In some embodiments, the organic fraction comprises about 90 wt. % free fatty acids and about 10 wt. % glycerine and/or glycerol. In some embodiments, the organic fraction comprises at least about 50 to about 100 wt. %, about 60 to about 100 wt. %, about 70 to about 100 wt. %, about 80 to about 100 wt. %, about 90 to about 100%, about 60 to about 90 wt. %, or about 70 to about 80 wt. % free fatty acids. In some embodiments, the separation of the saturated fatty acids from the unsaturated fatty acids in c) comprises temperature dependent solvent extraction. In some embodiments, the saturated free fatty acids of a) are separated into short-chain saturated free fatty acids, optionally C8-12 saturated free fatty acids, and long-chain saturated free fatty acids, optionally C13-C22, C15-C19, or C16-C22 saturated free fatty acids. In some embodiments, the method further includes decarboxylation of the long-chain fatty acids. In some embodiments, the decarboxylation comprises a catalyst selected from Mo on Al₂O₃, MgO on Al₂O₃, and Ni on Al₂O₃, optionally comprising a single-stage continuous process and/or subcritical water. In some embodiments, the ethenolysis comprises a catalyst, optionally selected from tungsten, molybdenum, rhenium and ruthenium. In some embodiments, the unsaturated free fatty acids comprise and/or consist of long-chain unsaturated free fatty acids. In some embodiments, the method further includes separating the alpha olefins from the short-chain unsaturated fatty acids by oligomerization, optionally in the presence of a heterogenous catalyst, optionally providing an alpha olefin dimer, an alpha olefin trimer, an alpha olefin tetramer, and/or an alpha olefin pentamer. In some embodiments, the heterogenous catalyst is selected from metals, metal oxides, metal salts, or organic materials (e.g. organic hydroperoxides, ion exchangers, and enzymes). In some embodiments, the method further includes isomerizing the alpha olefins, optionally in the presence of hydrogen or under inert conditions. In some embodiments, isomerization is performed inside a Parr reactor. In some embodiments, the temperature condition of isomerization reaction ranges from about 100° C. to about 500° C., about 100° C. to about 200° C., about 200° C. to about 300° C., about 300° C. to about 400° C., or about 400° C. to about 500° C. In some embodiments, the pressure condition of isomerization reaction ranges from about 1,000 psi to about 3,000 psi, from about 1,000 psi to about 2,000 psi, from about 2,000 psi to about 3,000 psi, or from about 1,500 psi to about 2,500 psi. In some embodiments, the heterogenous catalyst is selected from AlCl₃ and BF₃. In some embodiments, the glycerolysis produces short chain unsaturated acyl-glycerides. In some embodiments, the glycerolysis is base catalyzed, optionally wherein the catalyst a catalyst, optionally wherein the catalyst is a methoxide selected from sodium methoxide, potassium methoxide, lithium methoxide, zinc methoxide, calcium methoxide, tributyltin methoxide, magnesium methoxide, tantalum (V) methoxide, titanium (IV) methoxide, antimony (III) methoxide, germanium methoxide, copper (II) methoxide, and combinations thereof.

In aspects, the disclosure provides a method for preparing a base oil from a renewable oil comprising triglycerides comprising:
a) transesterifying the renewable oil to produce a mixture comprising;
   i) fatty acid ester mixture comprising;
      saturated fatty acid esters and unsaturated fatty acid esters, and
   ii) glycerin;
b) isolating the glycerin from the fatty acid ester mixture;
c) separating the saturated fatty acid esters from the unsaturated fatty acid esters;
d) subjecting the unsaturated fatty acid esters to ethenolysis to prepare a mixture comprising;
   i) alpha olefins; and
   ii) short-chain unsaturated fatty acid esters, optionally C6-C12 or C8-C12 short chain unsaturated fatty acid esters.

In some embodiments, in a), the renewable oil is purified prior to acidification. In some embodiments, purifying the renewable oils comprises clarification, degumming, bleaching, and/or filtering. In some embodiments, in a), transesterifying comprises reacting the renewable oil with an alcohol, optionally methanol, optionally in the presence of a catalyst. In some embodiments, the separation of the saturated fatty acid esters from the unsaturated fatty acid esters in c) comprises temperature dependent solvent extraction. In some embodiments, the saturated fatty acid esters of a) are separated into short-chain saturated fatty acid esters, optionally C8-12 saturated fatty acid esters, and long-chain saturated fatty acid esters, optionally C13-C22, C15-C19, or C16-C22 saturated fatty acid esters. In some embodiments, the method includes converting at least a portion of the short chain unsaturated fatty acid esters of d) into short chain unsaturated fatty acids; and decarboxylating the short chain unsaturated fatty acids of e) to produce saturated hydrocarbons, optionally wherein the decarboxylation comprises a catalyst or gas phase decarboxylation, optionally a Ni/C catalyst or an oxidative metal catalyst (e.g. silver (II)). In some embodiments, method further comprising: converting the long-chain fatty acid esters into long chain fatty acids; and decarboxylation of at least a portion of the long-chain fatty acids of g) to produce saturated hydrocarbons, optionally wherein the decarboxylation comprises a catalyst or gas phase decarboxylation, optionally a Ni/C catalyst or an oxidative metal catalyst (e.g. silver (II)). In some embodiments, the decarboxylation comprises a catalyst selected from Mo on Al₂O₃, MgO on Al₂O₃, and Ni on Al₂O₃, optionally comprising a single-stage continuous process and/or subcritical water. In some embodiments, the ethenolysis comprises a catalyst, optionally selected from tungsten, molybdenum, rhenium and ruthenium. In some embodiments, the unsaturated fatty acid esters comprise and/or consist of long-chain unsaturated fatty acid esters. In some embodiments, the method further includes separating the alpha olefins from the short-chain unsaturated fatty acid esters by oligomerization, optionally in the presence of a heterogenous catalyst, optionally providing an alpha olefin dimer, an alpha olefin trimer, an alpha olefin tetramer, and/or an alpha olefin pentamer. In some embodiments, the heterogenous catalyst is selected from metals, metal oxides, metal salts, or organic materials (e.g. organic hydroperoxides, ion exchangers, and enzymes). In some embodiments, the method further includes isomerizing the alpha olefins, optionally in the presence of hydrogen or under inert conditions. In some embodiments, isomerization is performed inside a Parr reactor. In some embodiments, the temperature condition of isomerization reaction ranges from about 100° C. to about 500° C., about 100° C. to about 200° C., about 200° C. to about 300° C., about 300° C. to about 400° C., or about 400° C. to about 500° C. In some embodiments, the pressure condition of isomerization reaction ranges from about 1,000 psi to about 3,000 psi, from about 1,000 psi to about 2,000 psi, from about 2,000 psi to about 3,000 psi, or from about 1,500 psi to about 2,500 psi. In some embodiments, the heterogenous catalyst is selected from AlCl₃ and BF₃. In some embodiments, the method further comprises combining the glycerin from a) with at least a portion of the short chain unsaturated fatty acids of e) to produce a mixture and subjecting the mixture to glycerolysis. In some embodiments, the glycerolysis produces short chain unsaturated acyl-glycerides. In some embodiments, the glycerolysis is base catalyzed, optionally wherein the catalyst a catalyst, optionally wherein the catalyst is a methoxide selected from sodium methoxide, potassium methoxide, lithium methoxide, zinc methoxide, calcium methoxide, tributyltin methoxide, magnesium methoxide, tantalum (V) methoxide, titanium (IV) methoxide, antimony (III) methoxide, germanium methoxide, copper (II) methoxide, and combinations thereof. In some embodiments, the renewable oil comprises or consists of one or more selected from seed oil, vegetable oil, and animal derived oils. In some embodiments, the renewable oil is selected from rapeseed oil, soy oil, castor oil. In some embodiments, the renewable oil is derived from one or more of poultry, beef, and fish.

In aspects, the disclosure provides a lubricant comprising:
a) a saturated hydrocarbon base oil in an amount ranging from about 50 wt % to about 70 wt % of the total weight of the lubricant, wherein the saturated hydrocarbon base oil comprises oligomers of C14-C18 olefin monomers, the dimers having an average carbon number in a range of from 29 to 36;
b) a viscosity modifier in an amount ranging from about 1 wt % to about 30 wt %, optionally about 1.4 wt %, about 1.80 wt %, about 3.2 wt %, about 4.13 wt %, about 5.2 wt %, about 16.25 wt %, or about 26 wt %, of the total weight of the lubricant;
c) a detergent in an amount ranging from about 10 wt % to about 15 wt %, optionally about 12.3 wt %, of the total weight of the lubricant; and
d) a pour point depressant in an amount ranging from about 0.1 wt % to about 1 wt %, optionally about 0.3 wt %, of the total weight of the lubricant.

In some embodiments, the saturated hydrocarbon base oil exhibits one or more of the following properties:
a) a Noack Volatility as measured by ASTM D5800 and/or CEC L-40-A-93 that is less than about 14%, less than about 13%, less than about 12%, less than about 11%, less than about 10%, or less than about 9%, optionally about 7.4%;
b) a Bromine Index below about 1000 mg Br2/100 g, about 500 mg Br2/100 g, or below about 200 mg Br2/100 g as determined in accordance with D2710-09;
c) an average branching index (BI) as determined by 1H NMR that is in the range of about 22 to about 26;
d) an average paraffin branching proximity (BP) as determined by 13C NMR in a range of from about 18 to about 26;
e) a viscosity index as determined in accordance with ASTM D2270 of about 125 or greater, about 130 or greater, about 135 or greater, or about 140 or greater;
f) a pour point as determined in accordance with ASTM D97 less than about −20° C., less than about −27° C., less than about −30° C., less than about −33° C., less than about −36° C., less than about −39° C., or less than about −42° C.;
g) a Cold Crank Simulated (CCS) dynamic viscosity as measured by ASTM D5293 at −35° C. less than about 1800 cP, less than about 1700 cP, less than about 1600 cP, less than about 1500 cP, less than about 1400 cP, less than about 1300 cP, less than about 1200 cP, or less than about 1100 cP; and
h) a KV (100) as measured by ASTM D445-17a that is in the range of about 3.7 cSt to about 9.7 cSt, or about 3.7 cSt to about 4.8 cSt.

In some embodiments, the saturated hydrocarbon base oil comprises SynNova 4 in an amount ranging from about 50 wt % to about 60 wt % of the total weight of the lubricant, and SynNova 9 in an amount ranging from about 3 wt % to about 7 wt % of the total weight of the lubricant. In some embodiments, the viscosity modifier comprises one or more of Infineum SV603 and Infineum SV261L, the detergent comprises Infineum P6003, and the pour point depressant comprises Infineum V385.

In some embodiments, the lubricant comprises:
a) a saturated hydrocarbon base oil comprising SynNova 4 in an amount ranging from about 56 wt % to about 57 wt % of the total weight of the lubricant, and SynNova 9 in an amount ranging from about 4.5 wt % to about 5.5 wt % of the total weight of the lubricant;
b) a viscosity modifier comprising Infineum SV603 in an amount ranging from about 25.5 wt % to about 26.5 wt % of the total weight of the lubricant;
c) a detergent comprising Infineum P6003 in an amount ranging from about 12 wt % to about 13 wt % of the total weight of the lubricant; and
d) a pour point depressant comprising Infineum V385 in an amount ranging from about 0.2 wt % to about 0.4 wt % of the total weight of the lubricant.

### BRIEF DESCRIPTION OF THE FIGURES

The drawings illustrate generally, by way of example, but not by way of limitation, various embodiments discussed in the present document.

FIG. 1 illustrates a flowchart showing a non-limiting pathway for the formation of renewable alpha olefins, renewable diesel, synthetic gasoline, and unsaturated acyl-glycerides from vegetable oil.

FIG. 2 illustrates a gas chromatography (GC) spectrum of a base oil prepared by the method of the disclosure. Carbon assignments are based on oligomerization of C16 alpha olefin (AO).

FIG. 3 shows an image of the process of degumming soy oil.

FIG. 4 shows an image of purified soy oil.

FIG. 5 shows an image of the acid hydrolysis of oil in process.

FIG. 6 shows an image of the oil post-hydrolysis.

FIG. 7 shows an image of an oxidative decarboxylation of fatty acids reactor.

FIG. 8 shows an image of an ethenolysis reactor.

FIG. 9 illustrates a gas chromatography (GC) spectrum of 1-decene yield.

FIG. 10 illustrates a gas chromatography (GC) spectrum for the ethenolysis of methyl oleate.

FIG. 11 illustrates a gas chromatography (GC) spectrum for the ethenolysis of oleic acid.

FIG. 12 illustrates a flowchart showing a non-limiting pathway for the formation of renewable alpha olefins, renewable diesel, synthetic gasoline, and unsaturated acyl-glycerides from vegetable oil, involving the conversion of soy to fatty acid esters using transesterification as a method to separate glycerin and provide pre-ethenolysis material.

FIG. 13 illustrates a gas chromatogram of soy oil sample.

FIG. 14 illustrates a gas chromatogram of a sample of product resulting from transesterification of a soy oil sample.

### DEFINITIONS

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

As used in the preceding sections and throughout the rest of this specification, unless defined otherwise, all technical and scientific terms used herein have the same meaning as is commonly understood by one of skill in the art to which this disclosure belongs. All patents and publications referred to herein are incorporated by reference in their entireties.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that section. A comma can be used as a delimiter or digit group separator to the left or right of a decimal mark; for example, “0.000,1” is equivalent to “0.0001.” All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods of manufacturing described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “organic group” as used herein refers to but is not limited to any carbon-containing functional group. For example, an oxygen-containing group such as an alkoxy group, aryloxy group, aralkyloxy group, oxo (carbonyl) group, a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O) CH₂C(O)R, C(S)R, C(O)OR, OC(O)R, C(O)N(R)₂, OC(O)N(R)₂, C(S)N(R)₂, (CH₂) 0-2N(R)C(O)R, (CH₂)₀₋₂N(R)N(R)₂, N(R)N(R)C(O)R, N(R)N(R)C(O) OR, N(R)N(R) CON(R)₂, N(R) SO₂R, N(R) SO₂N(R)₂, N(R)C(O) OR, N(R)C(O)R, N(R)C(S)R, N(R)C(O)N(R)₂, N(R) C(S)N(R)₂, N(COR) COR, N(OR)R, C(═NH)N(R)₂,C(O)N(OR)R, or C(═NOR)R, wherein R can be hydrogen (in examples that include other carbon atoms) or a carbon-based moiety, and wherein the carbon-based moiety can itself be further substituted.

The term “composition” as used herein refers to a chemical, compound, or substance, or a mixture or combination of two or more such chemicals, compounds, or substances.

The term “solvent” as used herein refers to a liquid that can dissolve a solid, another liquid, or a gas. Non-limiting examples of solvents are silicones, organic compounds, water, alcohols, ionic liquids, and supercritical fluids.

The term “room temperature” as used herein refers to a temperature of about 15° C. to about 28° C.

The term “standard temperature and pressure” as used herein refers to 20° C. and 101 kPa.

As used herein, the term “Long-chain free fatty acid” refers to unmodified free fatty acids that have either been hydrolysis and separated from their glycerol backbone or, still in acyl-glyceride form.

As used herein, the term “Short-chain free fatty acid” refers to unmodified free fatty acids that have either been subject to ethenolysis or form of molecular splitting of the free fatty acid or free fatty acid portion of an acyl-glyceride and separated from their glycerol backbone.

The term “olefin” as used herein refers a hydrocarbon containing at least one carbon-carbon double bond. For example, according to aspects of the disclosure herein, an olefin may comprise a hydrocarbon chain length of from C14 to C18, and may have a double bond at an end (primary position) of the hydrocarbon chain (alpha-olefin) or at an internal position (internal-olefin). In one embodiment, the olefin is a mono-olefin, meaning that the olefin contains only a single double-bond group.

The term “dimer” as used herein refers to molecules formed by the combination of two monomers via a chemical process, where in monomers may be the same or different type of monomer unit. The dimer may be formed by chemical reaction and/or other type of bonding between the monomers. In one embodiment, a dimer is the product of oligomerization between two olefin monomers.

The term “Dimer Total Average Carbon Number” is used herein to refer to a total number of carbons in the dimer. Accordingly, a “C29-C36” dimer as referred to herein is a dimer having a total average number of carbon atoms in a range of from 29 to 36.

The term “Trimer Total Average Carbon Number” is used herein to refer to a total number of carbons in the trimer. Accordingly, a “C45-C52” trimer as referred to herein is a dimer having a total average number of carbon atoms in a range of from 45 to 52.

The term “Tetramer Total Average Carbon Number” is used herein to refer to a total number of carbons in the tetramer. Accordingly, a “C61-C68” tetramer as referred to herein is a dimer having a total average number of carbon atoms in a range of from 61 to 68.

The term “Pentamer Total Average Carbon Number” is used herein to refer to a total number of carbons in the pentamer. Accordingly, a “C77-C84” pentamer as referred to herein is a dimer having a total average number of carbon atoms in a range of from 77 to 84.

### DETAILED DESCRIPTION

### Methods for Preparing Base Oils

In one aspect, the disclosure provides methods of preparing base oils, including but not limited to hydrocarbon base oils such as long and short-chain alpha olefins, saturated hydrocarbons, and acyl-glycerides, and lubricants comprising same.

In some embodiments, methods of preparing long-chain alpha olefins include oligomerization, isomerization, and hydrogenation of the long-chain alpha and internal olefins, which provides a viable alternative to petroleum-based lubricants. In some embodiments, when this product is combined with specific additive types, the resulting product may be capable of replacing synthetic, petroleum base feedstocks when creating lubricant base stock and/or base oil.

In some embodiments, unsaturated and saturated free fatty acid by-products provide an alternative for synthetic gasoline and renewable diesel via decarboxylation. In some embodiments, a secondary process for the formation of value-added acyl-glycerides from short-chain free fatty acids is also described in conjunction with the overall process, which can be useful as an alternative to synthetic gasoline.

One relatively new approach to the use of renewable oils is the pairing of technologies to produce valuable lubricant base stocks, as well as commercially available fuels like renewable diesel and synthetic gasoline. Converting a percent of the feedstock into a value-added by-product like alpha olefins for lubricant production, then selectively separating out a percentage for further conversion into biofuels like renewable diesel and synthetic gasoline, could help diversify manufacturing. Renewable oils that contain a combination of saturated and unsaturated free fatty acids are an ideal substrate for this process.

Using novel separation techniques, the saturated free fatty acids can be segregated and processed separately into renewable or “green” diesel. Green diesel (also referred to as renewable hydrocarbon diesel, hydro processed vegetable oils or HVO) is substantially the same chemically as petroleum-derived diesel, but green diesel is made from recently living biomass. Unlike biodiesel, which is an ester and has different chemical properties from petroleum diesel, green diesel is composed of long-chain hydrocarbons, and can be mixed with petroleum diesel in any proportion for use as transportation fuel.

The unsaturated free fatty acids are then sent on to produce both alpha and internal olefins and synthetic gasoline. Traditionally, olefins are produced from non-biogenic precursors, but one alternative to petroleum based alpha olefins is the conversion of unsaturated fatty acids to linear alpha olefins. This can be achieved through a process called “ethenolysis”, reacting molecular ethene with the double-bond on the carbon chain of a fatty acid molecule. The hydrocarbon chain is left with a terminal double bond on the last carbon in the chain, while the free fatty acid converts to a smaller chain designation. In the example of oleic acid (C18:1), the by-products are 1-decene (1-C10) and a C10 fatty acid. The remaining fatty acid can be decarboxylated to form another alpha olefin, 1-nonene (1-C9), which can then be used in the synthesis of synthetic gasoline.

Aspects of this present disclosure also relate to lubricants that include non-fossil based hydrocarbon base oils and have improved properties over petroleum-based lubricants.

Recent patents have shown various approaches to creating petroleum alternatives for fuels and lubricants. Two patent publications, US 2020/0165538 A1 and U.S. Pat. No. 10,961,167 B2, which are each incorporated by reference herein in their entireties, describe in detail the conversion of pristine alpha olefins by oligomerization, hydrogenation, and isomerization to make a lubricating oil base stock. Both patents describe the precursor alpha olefins to come from petroleum sources. A third patent, U.S. Pat. No. 9,862,906 B2, which is incorporated by reference herein in its entirety, discusses a similar process and product but utilized terpenes as the feedstock. In their current embodiment, these patents make no claim to using olefins derived from renewable sources.

The metathesis reaction ethenolysis applied to fatty acid esters has been reported in many publications and scientific reviews and is well known in organic chemistry. It is carried out in the presence of a catalyst and consists of exchanging alkylidene groups between two olefins, in this case the unsaturated free fatty acid and ethene. A patent publication US 2006/0079704 A1, which is incorporated by reference herein in its entirety, published in 2006, discusses the use of ethenolysis on the triglycerides to produce alpha olefins. The process describes the catalytic mechanism and various by-products of the reaction, with no discussion as to the use of the primary product alpha olefin or by-product glycerol.

The use of glycerol-esterification or glycerolysis as a pretreatment for methyl ester (biodiesel) production has become popular during the past decade. Its ability to reduce free fatty acid levels to below 0.2% wt make it attractive to methyl ester production, because free fatty acids will neutralize catalysts in downstream reactions. However, utilizing glycerolysis as a means of treating a ˜100% wt free fatty acid by-product stream, using recycled glycerol, is a new approach that requires the same chemical mechanism, but requiring significantly different reaction conditions and chemical loading.

The patents discussed above do not address utilizing any of the by-products generated during processing to help reduce the carbon intensity (C.I.) of the overall process. By staging the sequence of operation according to the disclosed patent, the claims in the patents referenced above can be synergistically improved to reduce the energy and logistical restraints of the independent technologies. Additionally, creating several petroleum alternatives in a single process allows for the claiming of government incentives like Renewable Identification Numbers (RINs), and in the case of California, Low-carbon Fuel Standard. Utilizing the process disclosure in the state of California at this time could significantly reduce the operational costs and process carbon intensity by collecting state and federal incentive funds.

### Purification of Renewable Oils

In one aspect, the disclosure provides a method for preparing a base oil from a renewable oil. Non-limiting examples of base oils that can be prepared by the methods of the disclosure include hydrocarbon base oils such as long and short-chain alpha olefins, saturated hydrocarbons, and acyl-glycerides.

In some embodiments, the method for preparing a base oil and/or base stock includes clarification, degumming, purification, and/or refining of a renewable oil. In some embodiments, the process described herein can utilize a broad range of renewable oils, including but not limited to seed and vegetable oils (rape, soy, castor, etc.), and animal derived oils (poultry, beef, fish, etc.). These oils collectively will be known as “renewable oils” for the purpose of this disclosure. In some embodiments, the renewable oil comprises triglycerides. In some embodiments, the renewable oil comprises and/or consists of soybean oil. Soybean oil can include C16-C22 fatty acids, including triglycerides comprising C16-C22 fatty acids, and can include about 20% oleic acids (C18:1) and about 55% linoleic acids (C18:2).

In some embodiments, the purification produces an oil precursor of high enough quality to continue through the lubricant process, not to optimize the filter-aid to oil ratio. The final material is then free of debris and unable to be separated via lab centrifugation.

In some embodiments, the process of purification of the renewable oils includes but is not limited to clarification, degumming, bleaching, and filtering. In some embodiments, purification provides renewable oil free of or substantially free of water, insolubles and/or unsaponifiables (MIU).

In some embodiments, the purification and/or refining of renewable oils involves a degumming step. In a non-limiting example, the degumming step includes using water and acid to remove phospholipids and other gums. Non-limiting examples of acids include citric acid. In some embodiments, the acid (e.g. citric acid) is at a concentration of about 1% to about 10% wt/wt, or about 4% to about 6% wt/wt in water. In some embodiments, the acid (e.g. citric acid) is at a concentration of about 5% wt/wt in water. In some embodiments, the degumming step includes heating a mixture comprising the renewable oil, water, and acid at a temperature ranging from about 50° C. to about 100° C., about 60° C. to about 70° C., or about 65° C. In some embodiments, free fatty acids are commonly neutralized using a base like sodium hydroxide, which is capable of producing a by-product stream of soap stock.

In some embodiments, the purification comprises a bleaching stage to remove color-bodies, polymeric compounds, free fatty acids, soaps, and/or trace metals. In some embodiments, the bleaching stage can be used to deodorize the oil and/or eliminate potential oxidation products.

In some embodiments, the purification comprises clarification. In some embodiments, clarification comprises separating solids from renewably sourced oils to provide a clarified oil. In some embodiments, separating the solids includes filtering the oil to provide a particulate-free or substantially particulate-free liquid. In some embodiments, filtering removes insoluble components from the oil (e.g., debris such as plastics, biomass particulate, and/or other impurities). Non-limiting examples of suitable filters include commercially available filtration aids such as diatomaceous earth, filter paper (e.g. 50 microns), cellulosic filter aid, filter bags at a combination of filter pore sizes.

### Transesterification of Renewable Oil to Produce Fatty Acid Esters and Glycerol

In one aspect, the method includes transesterification of the renewable oil. In some embodiments, the renewable oil comprises triglycerides. In some embodiments, the method comprises transesterifying the renewable oil to produce a mixture comprising a 1) fatty acid ester mixture comprising saturated fatty acids esters and unsaturated fatty acids esters, and 2) glycerin and/or glycerol. In some embodiments, the renewable oil is purified (e.g. clarified and/or degummed) prior to the transesterification.

Any transesterification method is contemplated by the present disclosure, as would be understood by one of ordinary skill in the art. In a non-limiting example, the renewable oil is dissolved in an alcohol (e.g. methanol) and transesterified, optionally under catalytic conditions, to produce glycerin and/or glycerol, and fatty acid esters (e.g. fatty acid methyl esters). Non-limiting examples of catalytic conditions useful for transesterification include acid catalysts (e.g. sulfonic acid, sulfuric acid, and trifluoroacetic acid), base catalysts (sulfonic and sulfuric acids), and enzymatic catalysts (e.g. lipases). In some embodiments, the catalyst is a metal (e.g. potassium metal). In a non-limiting example, the metal can form a metal alkoxide and/or alkoxylate with an alcohol (e.g. potassium methoxylate). In some embodiments, the catalyst is added in an amount ranging from about 1% (w/vol) to about 5% (w/vol), or about 1% (w/vol). In some embodiments, the transesterification is performed in the absence of a catalyst. Non-limiting examples of alcohols include methanol, ethanol, and n-propanol. In some embodiment, the alcohol comprises and/or consists of methanol.

In some embodiments, transesterification includes reacting the renewable oil (e.g. clarified and/or degummed renewable oil) with an alcohol (e.g. methanol), optionally in the presence of a catalyst. In some embodiments, the reaction further comprises the addition of water and an organic solvent (e.g. dichloromethane, n-hexanes, ethyl acetate) to provide an organic fraction comprising free fatty acid esters derived from the renewable oil, which is optionally purified (e.g. clarified and/or degummed), and an aqueous fraction comprising glycerin and/or glycerol (e.g. glycerin component).

In some embodiments, transesterification of the renewable oil (e.g. clarified and/or degummed renewable oil) comprises reacting the renewable oil with an alcohol (e.g. methanol), optionally in the presence of a catalyst. In some embodiments, the reaction further comprises the addition of water and an organic solvent (e.g. dichloromethane, n-hexanes, ethyl acetate) to provide an organic fraction and an aqueous fraction, wherein the organic fraction comprises the free fatty acid ester mixture and the aqueous fraction comprises the glycerin. In some embodiments, the transesterification to prepare an aqueous fraction and an organic fraction is repeated more than once. In some embodiment the organic fraction further includes chemicals, compounds, and/or substances that are relatively insoluble in water.

In some embodiments, a portion of the organic solvent can be separated from the organic fraction following transesterification to provide free fatty acid esters derived from the oil. In some embodiments, separating at least a portion of the organic solvent includes heating the organic fraction to a temperature of at least about 50° C., about 55° C., about 60° C., about 65° C., about 70° C., about 75° C., about 80° C., about 85° C., about 90° C., about 95° C., about 100° C., about 105° C., about 110° C., about 115° C., or about 120° C., exposing the organic fraction to a pressure of about 1 atm or less, or both. In some embodiments, the separation of the organic solvent from the organic fraction can be achieved, for example, by rotary evaporation or a similar technique.

In some embodiments the organic fraction includes at least about 90 wt. % fatty acid esters and about 10 wt. % glycerin and/or glycerol. In some embodiments the organic fraction includes 90 wt. % to about 100 wt % fatty acid esters and about 0 wt. % to about 10 wt. % glycerin and/or glycerol. In some embodiments the organic fraction includes at least about 50 wt. %, about 60 wt. %, about 70 wt. %, about 80 wt. % or about 90 wt. % fatty acid esters. In some embodiments, the organic fraction comprises about 50 to about 100 wt. %, about 60 to about 100 wt. %, about 70 to about 100 wt. %, about 80 to about 100 wt. %, about 90 to about 100%, about 60 to about 90 wt. %, or about 70 to about 80 wt. % fatty acid esters.

In some embodiments, the renewable oil and alcohol, optionally in the presence of a catalyst, are heated to a temperature ranging from about 50° C. to about 150° C., about 100° C. to about 125° C., or about 115° C.

In some embodiments, the transesterification of the renewable oil comprises heating a mixture of renewable oil and an alcohol at a suitable pressure. In some embodiments, the ratio of renewable oil to water ranges from about 5:1 to about 1:5, about 4:1 to about 1:4, about 3:1 to about 1:3, or about 2:1 to about 1:2 based on the total weight of the renewable oil and alcohol. In some embodiments, the ratio of renewable oil to alcohol is about 5:1, about 4:1, about 3:1, about 2:1, or about 1:1 based on the total weight of the renewable oil and alcohol.

In some embodiments, the renewable oil and alcohol are heated to a temperature ranging from about 100° C. to about 350° C., about 200° C. to about 300° C., about 250° C. to about 275° C., or a temperature of about 250° C., about 255° C., about 260° C., about 265° C., about 270° C., or about 275° C.

In some embodiments, transesterification of the renewable oil is performed at a pressure ranging from about 500 psi to about 1000 psi, about 700 psi to about 900 psi, or about 800 psi to about 900 psi. In some embodiments, acidifying and/or acidulating the renewable oil is performed at a pressure of about 800 psi, about 810 psi, about 820 psi, about 830 psi, about 840 psi, about 850 psi, about 860 psi, about 870 psi, about 880 psi, about 890 psi, or about 900 psi.

In some embodiments, transesterification of the renewable oil is performed in a reaction vessel including but not limited to a high-temperature and/or high-pressure mixed reactor. In some embodiments, the reaction vessel is purged with an inert gas (e.g. nitrogen) after the renewable oil and water are added to the reaction vessel. In a non-limiting example, purging the reactor with an inert gas prevents unwanted oxidation during the acidifying and/or acidulating.

In some embodiments, the method comprises isolating the glycerin and/or glycerol from the mixture of fatty acid esters. In some embodiments, the organic fraction further separates into a denser organic phase and a lighter organic phase. In some embodiments, the glycerin and/or glycerol phase produced from the reaction is allowed to gravity separate to the bottom of the reactor vessel, optionally forming part of the denser organic phase. In some embodiments, the denser organic phase can be separated via methods such as, but not limited to, mechanical clarification or centrifugation. In some embodiments, the lighter organic phase is composed primarily of fatty acid esters and is separated from the denser organic phase, which comprises glycerin and/or glycerol.

In some embodiments, transesterifying the renewable oil, optionally purified renewable oil (e.g. clarified and/or degummed) further includes separating at least a portion of the aqueous fraction from the organic fraction before converting at least the portion of the fatty acid esters to glycerides (e.g. acyl glycerides).

In some embodiments, the organic solvent is chosen from at least one of hexane, diethyl ether, ethyl acetate, and dichloromethane. In some embodiments, separation after transesterification further includes recycling the separated portion of the organic solvent to be used for contact with the renewable oil (e.g. clarified renewable oil).

In some embodiments, the glycerine and/or glycerol, and any alcohol (e.g. methanol) may be recycled into a downstream process known as glycerolysis.

In some embodiments, transesterification comprises heating the renewable oil (e.g. clarified and/or degummed renewable oil) further includes heating the renewable oil (e.g. clarified and/or degummed renewable oil) and an alcohol. In some embodiments, heating can occur at a pressure of about 1 atm to about 3 atm, or about 1 atm, 2 atm, or 3 atm.

In some embodiments, the glycerin and/or glycerol is dried at a temperature ranging from about 50° C. to about 200° C., or about 100° C. to about 125° C., or about 115° C. and/or at a pressure ranging from about 40 mmHg to about 100 mmHg, or about 50 mmHg to about 70 mmHg, or about 60 mmHg and/or for a time period ranging from about 5 minutes to about 2 hours, about 15 minutes to about 1 hour, or about 20 minutes.

In some embodiments, the fatty acid esters are dried at a temperature ranging from about 50° C. to about 200° C., or about 100° C. to about 125° C., or about 115° C. and/or at a pressure ranging from about 40 mmHg to about 100 mmHg, or about 50 mmHg to about 70 mmHg, or about 60 mmHg and/or for a time period ranging from about 5 minutes to about 2 hours, about 15 minutes to about 1 hour, or about 20 minutes.

### Acid Hydrolysis of Renewable Oil to Produce Free Fatty Acids and Glycerol

In some embodiments, the process includes acid hydrolysis (e.g. acidification and/or acidulation) the renewable oil. In some embodiments, the renewable oil comprises triglycerides. In some embodiments, the method comprises acidifying the renewable oil to produce a mixture comprising a 1) free fatty acid mixture comprising saturated fatty acids (e.g. free fatty acids) and unsaturated fatty acids (e.g. free fatty acids), and 2) glycerin and/or glycerol. In some embodiments, the renewable oil is purified (e.g. clarified and/or degummed) prior to the acid hydrolysis (e.g. acidification and/or acidulation).

In some embodiments, acidifying and/or acidulating includes contacting the renewable oil with an aqueous acid to provide an organic fraction comprising free fatty acids derived from the renewable oil, which is optionally purified (e.g. clarified and/or degummed) and an aqueous fraction containing the acid and glycerol phrase (glycerin component).

In some embodiments, acidifying and/or acidulating the renewable oil (e.g. clarified and/or degummed renewable oil) comprises contacting the renewable oil with an aqueous acid to form an organic fraction and an aqueous fraction, wherein the organic fraction comprises the free fatty acids mixture and the aqueous fraction comprises the glycerin. In some embodiments, the acidification to prepare an aqueous fraction and an organic fraction is repeated more than once. In some embodiment the organic fraction further includes chemicals, compounds, and/or substances that are relatively insoluble in water.

In some embodiments, the acid includes at least one of H₂SO₄, HCl, H₂PO₄, and H₃PO₄. In some embodiments, the acid is added at about 1 wt % to about 10 wt. %, or about 3 wt. % to about 5 wt. %. In some embodiments, the acid is added at about 4 wt %. In some embodiments, the acid is H₂PO₄, optionally at about 4 wt %. In some embodiments, the acid is H₂SO₄, optionally at about 4 wt %. In some embodiments the organic fraction includes at least about 90 wt. % free fatty acids and about 10 wt. % glycerin and/or glycerol. In some embodiments the organic fraction includes 90 wt. % to about 100 wt % free fatty acids and about 0 wt. % to about 10 wt. % glycerin and/or glycerol. In some embodiments the organic fraction includes at least about 50 wt. %, about 60 wt. %, about 70 wt. %, about 80 wt. % or about 90 wt. % free fatty acids. In some embodiments, the organic fraction comprises about 50 to about 100 wt. %, about 60 to about 100 wt. %, about 70 to about 100 wt. %, about 80 to about 100 wt. %, about 90 to about 100%, about 60 to about 90 wt. %, or about 70 to about 80 wt. % free fatty acids.

In some embodiments, after contacting the renewable oil with an aqueous acid further, the mixture is heated to a temperature ranging from about 50° C. to about 150° C., about 100° C. to about 125° C., or about 115° C.

In some embodiments, the acidifying and/or acidulating the renewable oil comprises heating a mixture of renewable oil and water at a suitable pressure. In some embodiments, the ratio of renewable oil to water ranges from about 5:1 to about 1:5, about 4:1 to about 1:4, about 3:1 to about 1:3, or about 2:1 to about 1:2 based on the total weight of the renewable oil and water. In some embodiments, the ratio of renewable oil to water is about 5:1, about 4:1, about 3:1, about 2:1, or about 1:1 based on the total weight of the renewable oil and water.

In some embodiments, the mixture is heated to a temperature ranging from about 100° C. to about 350° C., about 200° C. to about 300° C., about 250° C. to about 275° C., or a temperature of about 250° C., about 255° C., about 260° C., about 265° C., about 270° C., or about 275° C.

In some embodiments, acidifying and/or acidulating the renewable oil is performed at a pressure ranging from about 500 psi to about 1000 psi, about 700 psi to about 900 psi, or about 800 psi to about 900 psi. In some embodiments, acidifying and/or acidulating the renewable oil is performed at a pressure of about 800 psi, about 810 psi, about 820 psi, about 830 psi, about 840 psi, about 850 psi, about 860 psi, about 870 psi, about 880 psi, about 890 psi, or about 900 psi.

In some embodiments, acidifying and/or acidulating the renewable oil is performed in a reaction vessel including but not limited to a high-temperature and/or high-pressure mixed reactor. In some embodiments, the reaction vessel is purged with an inert gas (e.g. nitrogen) after the renewable oil and water are added to the reaction vessel. In a non-limiting example, purging the reactor with an inert gas prevents unwanted oxidation during the acidifying and/or acidulating.

In some embodiments, the method comprises isolating the glycerin and/or glycerol from the mixture of fatty acids. In some embodiments, the organic fraction further separates into a denser organic phase and a lighter organic phase. In some embodiments, the glycerin and/or glycerol phase produced from the reaction is allowed to gravity separate to the bottom of the reactor vessel, optionally forming part of the denser organic phase. In some embodiments, the denser organic phase can be separated via methods such as, but not limited to, mechanical clarification or centrifugation. In some embodiments, the lighter organic phase is composed primarily of free fatty acid and is separated from the denser organic phase, which is a mixture of acid, water, and glycerin.

In some embodiments, acidifying the renewable oil, optionally purified renewable oil (e.g. clarified and/or degummed) further includes separating at least a portion of the aqueous fraction from the organic fraction before converting at least the portion of the free fatty acids to glycerides (e.g. acyl glycerides).

In some embodiments, acidifying the renewable oil, (e.g. clarified and/or degummed renewable oil) includes contacting the material with an aqueous acid and an organic solvent to provide an organic fraction comprising a mixture of free fatty acids and an aqueous fraction. In some embodiments, a portion of the organic solvent can be separated from the organic fraction following the acidification separation to provide the acidified composition comprising free fatty acids derived from the oil. In some embodiments, separating at least a portion of the organic solvent includes heating the organic fraction to a temperature of at least about 50° C., about 55° C., about 60° C., about 65° C., about 70° C., about 75° C., about 80° C., about 85° C., about 90° C., about 95° C., about 100° C., about 105° C., about 110° C., about 115° C., or about 120° C., exposing the organic fraction to a pressure of about 1 atm or less, or both. In some embodiments, the separation of the organic solvent from the organic fraction can be achieved, for example, by rotary evaporation or a similar technique.

In some embodiments, the organic solvent is chosen from at least one of hexane, diethyl ether, ethyl acetate, and dichloromethane. The organic solvent can be obtained from a previous acidification separation. In some embodiments, acidification separation further includes recycling the separated portion of the organic solvent to be used for contact with the renewable oil (e.g. clarified renewable oil). In some embodiments, the aqueous acid comprises at least one of H₂SO₄, HCl, and H₃PO₄.

In some embodiments, acidifying the renewable oil (e.g. clarified and/or degummed renewable oil) includes contacting the renewable oil with the aqueous acid, methanol, and glycerol. In some embodiments, the methanol and glycerol may be recycled into a downstream process known as glycerolysis.

In some embodiments, acidifying the clarified renewable oil further includes heating the renewable oil (e.g. clarified and/or degummed renewable oil) and the aqueous acid. In some embodiments, heating the material can occur at a pressure of about 1 atm to about 3 atm, or about 1 atm, 2 atm, or 3 atm.

In some embodiments, the glycerin and/or glycerol is dried at a temperature ranging from about 50° C. to about 200° C., or about 100° C. to about 125° C., or about 115° C. and/or at a pressure ranging from about 40 mmHg to about 100 mmHg, or about 50 mmHg to about 70 mmHg, or about 60 mmHg and/or for a time period ranging from about 5 minutes to about 2 hours, about 15 minutes to about 1 hour, or about 20 minutes.

In some embodiments, the fatty acids (e.g. free fatty acids) are dried at a temperature ranging from about 50° C. to about 200° C., or about 100° C. to about 125° C., or about 115° C. and/or at a pressure ranging from about 40 mmHg to about 100 mmHg, or about 50 mmHg to about 70 mmHg, or about 60 mmHg and/or for a time period ranging from about 5 minutes to about 2 hours, about 15 minutes to about 1 hour, or about 20 minutes.

Solvent Extraction and Separation of Saturated and Unsaturated Fatty Acids and/or Saturated and Unsaturated Fatty Acid Esters

In one aspect, the process includes purifying and/or separating the fatty acids (e.g. free fatty acids) produced during acid hydrolysis into a fraction comprising: saturated free fatty acids and a fraction comprising unsaturated free fatty acids.

In another aspect, the process includes purifying and/or separating the fatty acid esters produced during transesterification into a fraction comprising: saturated fatty acid esters and a fraction comprising unsaturated fatty acid esters.

In some embodiments, organic solvents such as, but not limited to acetone are used to solubilize the unsaturated free fatty acids and/or unsaturated fatty acid esters while the saturated portion was precipitated out at low temperatures. In some embodiments, the degree of purification is dependent upon time, temperature, and/or stoichiometric equivalence of the organic solvent (e.g. acetone) to saturated free fatty acid and/or saturated fatty acid esters. In some embodiments, the separation of the saturated fatty acids from the unsaturated fatty acids and/or the saturated fatty acid esters from the unsaturated fatty acid esters comprises temperature dependent solvent extraction.

In some embodiments, the reaction was conducted at a temperature of about 55° C., about 60° C., or about 65° C.

In some embodiments, the temperature varied from −5 to +5 degrees Celsius during the precipitation reaction.

In some embodiments, the reactor mixture is agitated slowly. In some embodiments the mixture is static and unmixed. In some embodiments, the reactor mixture was agitated for about 15 minutes, for about 20 minutes, or for about 25 minutes. In some embodiments, the time during cooling and precipitation is varied to maximize the solubility and ultimately purification of the saturated from the unsaturated free fatty acids streams and/or saturated from the unsaturated fatty acid esters streams. In some embodiments, the amount of solvent is varied from 1:1 stoichiometric equivalence of organic solvent (e.g. acetone) to saturated free fatty acid, up to a ratio of 100:1 stoichiometric equivalence of organic solvent (e.g. acetone) to saturated free fatty acid; and/or 1:1 stoichiometric equivalence of organic solvent (e.g. acetone) to saturated fatty acid esters, up to a ratio of 100:1 stoichiometric equivalence of organic solvent (e.g. acetone) to saturated fatty acid esters.

In some embodiments, once the reaction is complete, the reactor contents are chilled in a beaker to about −5° C., about −4° C., about −3° C., about −2° C., about −1° C., about 0° C., about 1° C., about 2° C., about 3° C., about 4° C. or about 5° C. In some embodiments, the reactor contents are chilled in ethylene glycol and water baths. In some embodiments, the reactor contents are chilled for about 24 hours.

In some embodiments, the result is two phases including a solid precipitate containing highly concentrated levels of saturated free fatty acids and/or saturated fatty acid esters and a liquid phase containing a mixture of unsaturated free fatty acids and/or unsaturated fatty acid esters and acetone. In some embodiments, the solid phase produced from the reaction is separated from the liquid phase through methods including but not limited to filtration and/or centrifugation. In some embodiments, the organic solvent (e.g. acetone) is removed using a rotary evaporator, optionally under vacuum.

In some embodiments, the saturated free fatty acids are further separated into long-chain, saturated fatty acids (e.g. long-chain saturated free fatty acids) and short-chain, saturated fatty acids (e.g. short-chain saturated free fatty acids). In a non-limiting embodiment, long-chain saturated fatty acids comprise and/or consist of C13-C22, C15-C19, or C16-C22 fatty acids. In a non-limiting embodiment, short-chain saturated fatty acids comprise and/or consist of C6-C12 or C8-C12 fatty acids.

In some embodiments, the saturated fatty acid esters are further separated into long-chain, saturated fatty acid esters and short-chain, saturated fatty acid esters. In a non-limiting embodiment, long-chain saturated fatty acid esters comprise and/or consist of C13-C22, C15-C19, or C16-C22 fatty acid esters. In a non-limiting embodiment, short-chain saturated f fatty acid esters comprise and/or consist of C6-C12 or C8-C12 fatty acid esters.

### Decarboxylation of Saturated Fatty Acids

In one aspect, the method includes decarboxylation of saturated fatty acids (e.g. long-chain, saturated free fatty acids). In a non-limiting embodiment, long-chain saturated fatty acids comprise and/or consist of C13-C22, C15-C19, or C16-C22 fatty acids. In some embodiments, decarboxylation of long-chain, saturated fatty acids (e.g. long-chain, saturated free fatty acids) provides saturated hydrocarbons. In some embodiments, the decarboxylated long-chain, saturated fatty acids (e.g. saturated hydrocarbons) are useful as renewable diesel precursor. In some embodiments, the process comprises preparing renewable diesel. In some embodiments, the following the separation of saturated of long-chain, saturated free fatty acids, the process comprises removal of the terminal carboxyl function (e.g. decarboxylation) group. In some embodiments, the long-chain, saturated fatty acids (e.g. long-chain, saturated free fatty acids) are decarboxylated to produce renewable diesel fuel.

In some embodiments, saturated fatty acid esters are converted into saturated fatty acids (e.g. by saponification) which can then be decarboxylated. Non-limiting examples of methods for converting saturated fatty acid esters into saturated fatty acids includes treatment with aqueous alkali (e.g. NaOH).

Non-limiting examples of catalysts useful for decarboxylation include Mo on Al₂O₃, MgO on Al₂O₃, and Ni on Al₂O₃. In a non-limiting example, a single-stage continuous process, a catalyst, including but not limited to Mo on Al₂O₃, MgO on Al₂O₃, and Ni on Al₂O₃ was used and/or subcritical water was employed within the decarboxylation. In some embodiments, straight-chain hydrocarbons are obtained via decarboxylation and hydrogenation reactions with no added hydrogen. Mo/Al₂O₃ catalyst was found to exhibit a higher degree of decarboxylation and liquid yield compared to the other two examined catalysts (MgO/Al₂O₃, Ni/Al₂O₃) at the maximized conditions of 375° C., 4 h of space time, and a volume ratio of 5:1 of water to oleic acid. The obtained liquid product has a similar density (0.85 kg/m³ at 15.6° C.) and high heating value (44.7 MJ/kg) as commercial fuels including kerosene (0.78-0.82 kg/m³ and 46.2 MJ/kg), jet fuel (0.78-0.84 kg/m³ and 43.5 MJ/kg), and diesel fuel (0.80-0.96 kg/m³ and 44.8 MJ/kg). The reaction conditions including temperature, volume ratio of water-to-feed, and space time were maximized for the Mo/Al₂O₃ catalyst. Characterization of the spent catalysts showed that a significant amount of amorphous carbon deposited on the catalyst could be removed by simple carbon burning in air with the catalyst recycled and reused.

In some embodiments, residual solvent is removed by flash evaporation under vacuum pressure, prior to decarboxylation and loss of CO₂.

In some embodiments, the composition of carbon chain length of the long chain saturated free fatty acids ranges from C13-C22, C15-C19, or C16-C22, depending on the character of the incoming oil. The percent mass of saturated free fatty acids recovered is also dependent upon the character of the incoming oil.

In some embodiments, the saturated free fatty acids are decarboxylated, removing the terminal carboxylic acid function group from the saturated carbon chain. In some embodiments, the reaction is carried out in the presence of non-noble, metallic catalysts, such as Ni/C, Pt/C, followed up with hydrogenation.

In some embodiments, gas phase decarboxylation of hydrolyzed free fatty acid (FFA) has been investigated in two fixed-bed reactors by changing reaction parameters such as temperatures, FFA feed rates, and H₂-to-FFA molar ratios. FFA, which contains mostly C18 as well as a few C16, C20, C22, and C24 FFA, was fed into the boiling zone, evaporated, carried by hydrogen flow at the rate of 0.5-20 ml/min, and reacted with the 5% Pd/C catalyst in the reactor.

### Ethenolysis

In one aspect, the method includes subjecting unsaturated fatty acids (e.g. unsaturated free fatty acids) and/or unsaturated fatty acid esters to ethenolysis. In some embodiments, ethenolysis of the unsaturated fatty acids (e.g. unsaturated free fatty acids) provides a mixture comprising i) alpha olefins and ii) short-chain unsaturated fatty acids. In some embodiments, ethenolysis of the unsaturated fatty acid esters provides a mixture comprising i) alpha olefins and ii) short-chain unsaturated fatty acid esters. In some embodiments, the short-chain unsaturated fatty acids comprise and/or consist of C8-C12 short chain unsaturated fatty acids. In some embodiments, the short-chain unsaturated fatty acid esters comprise and/or consist of C8-C12 short chain unsaturated fatty acid esters. In some embodiments, the unsaturated fatty acids (e.g. unsaturated free fatty acids) are separated from saturated free fatty acids in the free fatty acid mixture prepared during acidification. In some embodiments, the unsaturated fatty acid esters are separated from saturated fatty acid esters in the fatty acid ester mixture prepared during transesterification. In some embodiments, the unsaturated free fatty acids comprise and/or consist of long-chain unsaturated free fatty acids. In some embodiments, the unsaturated fatty acid esters comprise and/or consist of long-chain unsaturated fatty acid esters. In a non-limiting example, following the separation of saturated and unsaturated long-chain fatty acids (e.g. saturated and unsaturated long-chain free fatty acids) and/or saturated and unsaturated long-chain fatty acid esters, a portion of long-chain, unsaturated free fatty acids and/or unsaturated long-chain fatty acid esters contained within the liquid phase post solvent extraction were segregated for further processing into terminal alpha olefins and unsaturated short-chain free fatty acids and/or unsaturated short-chain fatty acid esters.

In one aspect, the olefin metathesis reaction known commonly as ethenolysis is an equilibrated reaction. In some embodiments, it may occur in the presence of a wide variety of catalysts, usually based on transition metals from groups IVA to VIII, including but not limited to, tungsten, molybdenum, rhenium and ruthenium, either in the homogeneous phase and/or in the heterogeneous phase.

Various types of catalyst have been described for carrying out this transformation. In some embodiments, the first systems were homogeneous, based on tungsten and tetraalkyl tins, for example WCI/SnMea. In some embodiments, this was followed by heterogeneous systems based on rhenium activated by tetra alkyl tins. In some embodiments, complexes based on ruthenium have rapidly proved themselves to be very interesting because of their tolerance of a wide range of functional groups. In some embodiments, that property, coupled with an activity which is often high, explains their major development in the field of polymer synthesis and in organic synthesis. In some embodiments, their use to catalyze the metathesis of vegetable oils has been studied widely. The following references can be provided: International patent application WO-A-96/04289 (R. Grubbs et al) describes the ethenolysis of methyl oleate. In the presence of excess ethylene (100 psi), the reaction produces a mixture of decenes (43%), methyl decanoate. Additional non-limiting examples of catalysts include Grubbs' catalysts (ruthenium carbine complexes) and Schrock alkylidenes catalysts (molybdenum (VI) and tungsten (VI)-based catalysts). In some embodiments, the catalysts is Grubbs (I) catalyst (benzylidene-bis(tricyclohexylphosphine)-dichlororuthenium) and Grubbs (II) catalyst (benzylidene [1,3-bis(2,4,6-trimethylphenyl)-2-imidazolidinylidene]dichloro (tricyclohexylphosphine) ruthenium).

In some embodiments, the amount of catalyst used ranges from about 1% to about 10%, about 2% to about 5% based on the total weight of the saturated fatty acid.

In some embodiments, ethenolysis is performed in a non-ionic solvent. Non-limiting examples of non-ionic solvents include dichloromethane, n-hexane, and isooctane. In some embodiments, ethenolysis is performed in an ionic solvent. Non-limiting examples of ionic solvents include 1,1,3,3-tetramethylguanidium lactate [TMG][L], monoethanolammonium lactate [MEA][L], i-butyl-3-methylimidazolium tetrafluoroborate [BMIm][BF₄], i-butyl-3-methylimidazolium methylsulfate [BMIm][MeSO₄], i-hexyl-3-methylimidazolium methylsulfate [HMIm][MeSO₄], i-ethyl-3-methylimidazolium methylsulfate [EMIm][MeSO₄], and i-butyl-3-methylimidazolium hexafluorophosphate [BMIm][PF₆].

In some embodiments, the purified unsaturated fatty acids (e.g. unsaturated free fatty acids) and/or unsaturated fatty acid esters are combined with a non-ionic solvents (eg. dichloro-methane or n-hexane), a heterogeneous catalyst, and/or gaseous ethylene. In some embodiments the reaction temperature is about 30° C., about 35° C., about 40° C., about 45° C., or about 50° C. In some embodiments the reaction pressure is about 5 bar, about 6 bar, about 7 bar, about 8 bar, about 9 bar, about 10 bar, about 11 bar, about 12 bar, about 13 bar, about 14 bar, or about 15 bar.

In one aspect, if metathesis of the resulting unsaturated fatty acids (e.g. long-chain, unsaturated free fatty acids) by ethenolysis is applied not to a single chain of fatty acid, for example oleic or linoleic acid as above, but to a mixture of said fatty acid chains, as is the case when products are of vegetable or animal origin, a mixture of products will be obtained. In one aspect, if metathesis of the resulting unsaturated fatty acid esters (e.g. long-chain, unsaturated fatty acid esters) by ethenolysis is applied not to a single chain of fatty acid ester, for example methyl oleate or methyl linoleate, but to a mixture of fatty acid esters, a mixture of products will be obtained. In some embodiments, the nature of the products obtained, and their quantity will thus depend on the fatty acid and/or fatty acid ester composition of the fatty starting material used. In some embodiments, obtaining products which are rich in 1-decene implies using a starting material which is rich in oleic acid esters and/or oleic acids. In some embodiments, these oils are characterized by at least their fatty acid composition, by the nature and proportion of their unsaturated fatty acids. In some embodiments, at least about 80% of the fatty acid chains and/or fatty acid ester chains comprise oleic chains, the amount of linoleic fatty chains does not exceed 12% and the amount of linolenic fatty chains does not exceed about 0.3%. In some embodiments, other olefinic chains are present in said oils in an amount of more than about 0.3% while the amount of saturated chains, for example palmitic or stearic, is in the range of about 5% to about 15%.

In some embodiments, the resulting unsaturated fatty acids (e.g. long-chain, unsaturated free fatty acids) are reacted with ethylene in a metathesis reaction in the presence of at least one non-aqueous ionic liquid to produce both an olefinic fraction and a composition of mono-alcohols and short-chain free fatty acids. In some embodiments, the resulting unsaturated fatty acid esters (e.g. long-chain, unsaturated fatty acid esters) are reacted with ethylene in a metathesis reaction in the presence of at least one non-aqueous ionic liquid to produce both an olefinic fraction and a composition of mono-alcohols and short-chain free fatty acid esters.

In some embodiments, metathesis of renewable oils with ethylene used in excess may be carried out in a closed (batch) system, a semi-open system, or a continuous system with one or more reaction steps. It is also possible to carry out the reaction using reactive distillation. Vigorous agitation ensures good contact between the reagents (gas and liquid) and the catalytic mixture.

In some embodiments, the reaction temperature may be in the range of about 0° C. to about 150° C., or in the range of about 20° C. to about 120° C., or in the range of about 25° C. to about 50°, or in the range of about 25° C. to about 40° C. The operation may be carried out above or below the melting temperature of the medium, the dispersed solid state not being a limitation on the reaction. The pressure may, for example, be in the range from atmospheric pressure (about 0.1 MPa) to 50 MPa. In some embodiments, the ethylene may be used pure or as a mixture or diluted with a paraffin (inert).

In some embodiments, the reaction products may be separated by decanting. In some embodiments, it is also possible to separate the products by distillation if the ionic liquid is non-volatile and thermally stable.

### Hydroisomerization of Alpha Olefins

In one aspect, the method comprises isomerizing (e.g. hydroisomerizing) alpha olefins of the disclosure. In some embodiments, isomerization of alpha olefins provides compounds useful as base stock and/or base oil for lubricants. Isomerization is defined as the transformation of a molecule into a different isomer.

In some embodiments, the alpha olefins are separated from short-chain free fatty acids (FFA) to arrive at a structure desirable for use in lubricants. In some embodiments, co-solvent extraction and/or fractional distillation are used for separation. In some embodiments, differences in polarity and specific gravity between the two majority components (alpha olefins and short-chain FFAs) may result in an innate phase separation, avoiding the need for additional chemical treatments.

In some embodiments, after separating the alpha olefin portion, it is oligomerized in the presence of a heterogenous catalyst which includes one or more catalysts selected from metals, metal oxides, metal salts, or organic materials like organic hydroperoxides, ion exchangers, and enzymes. In some embodiments, the heterogeneous catalyst is one or more of AlCl₃ or BF₃. In some embodiments, post oligomerization analysis is used to confirm the degree of reaction. In some embodiments, the alpha olefins are separated from the short-chain unsaturated fatty acids by oligomerization. In some embodiments, oligomerization provides an alpha olefin dimer, an alpha olefin trimer, an alpha olefin tetramer, an alpha olefin pentamer, and mixtures thereof. In some embodiments, once the oligomerization (e.g. dimerization, trimerization, tetramerization, pentamerization) reaches published levels, the material is ready to be isomerized. Non-limiting methods useful for isomerization include the presence of hydrogen and a catalyst (e.g. Pd/C) or under inert conditions. In some embodiments, the purpose of isomerization is to increase the terminal branching character to the dimerized alpha olefin, while the hydrogen is used to saturate any residual double-bond character. In some embodiments, isomerization and hydrogenation can be performed inside a reactor, optionally with the degree of isomerization being controlled by the heterogenous catalyst type, temperature, pressure, and residence time. In some embodiments, isomerization is performed inside a Parr reactor.

In some embodiments, temperature conditions for isomerization reactions can range from about 100° C. to about 500° C., about 100° C. to about 200° C., about 200° C. to about 300° C., about 300° C. to about 400° C., or about 400° C. to about 500° C., optionally in the presence of a catalyst, for example a heterogenous catalyst.

In some embodiments, pressure conditions for isomerization reactions can range from about 1,000 psi to about 3,000 psi, from about 1,000 psi to about 2,000 psi, from about 2,000 psi to about 3,000 psi, or from about 1,500 psi to about 2,500 psi, optionally in the presence of heterogenous catalysts. In some embodiments, catalyst loading, temperatures, and/or pressure parameters are published and can be developed for lab applications.

### Decarboxylation of Unsaturated Fatty Acids

In one aspect, the method comprises decarboxylating unsaturated fatty acids (e.g. short-chain unsaturated fatty acids prepared from ethenolysis of unsaturated free fatty acids, including long-chain unsaturated free fatty acids). In some embodiments, decarboxylating unsaturated fatty acids produces saturated hydrocarbons. In some embodiments, following the ethenolysis of the long-chain, unsaturated free fatty acids, the primary product is an alpha olefin (e.g. linear alpha olefin), while the second by-product is a short-chain, unsaturated free fatty acid. In some embodiments, the short-chain unsaturated free fatty acids that resulted from the ethenolysis of unsaturated long-chain fatty acids were separated from the alpha olefin position via binary distillation. In some embodiments, the now concentrated short-chain, unsaturated free fatty acids are decarboxylated, removing the terminal carboxylic acid function group from the saturated carbon chain. In some embodiments, the composition of carbon chain length of the unsaturated free fatty acids ranges from C6-C12 or C8-C12, depending on the character of the incoming oil. In some embodiments, the percent mass of saturated free fatty acids recovered is also dependent upon the character of the incoming oil.

In some embodiments, unsaturated fatty acid esters are converted into unsaturated fatty acids (e.g. by saponification) which can then be decarboxylated. Non-limiting examples of methods for converting unsaturated fatty acid esters into unsaturated fatty acids includes treatment with aqueous alkali (e.g. NaOH).

In some embodiments, decarboxylation of fatty acids over non-noble metal catalysts without added hydrogen was studied. Non-limiting examples of catalysts useful for decarboxylation include Mo on Al₂O₃, MgO on Al₂O₃, Ni on Al₂O₃, and oxidazing metals (e.g. silver (II), which can be prepared in situ from silver nitrate and sodium persulfate). For a non-limiting example of decarboxylation of unsaturated fatty acids using silver (ii)-catalyzed oxidative decarboxylation, see van der Kils et al., Eur. J. Lipid Sci. Tech. 113:562-571 (2011), which is incorporated by reference herein in its entirety. In some embodiments, Ni/C catalysts were prepared and exhibited excellent activity and maintenance for decarboxylation. In some embodiments thereafter, the effects of nickel loading, catalyst loading, temperature, and carbon number on the decarboxylation of fatty acids were investigated. In some embodiments, the results indicate that the products of cracking increased with high nickel loading or catalyst loading. In some embodiments, temperature significantly impacted the conversion of stearic acid but did not influence the selectivity. In some embodiments, the fatty acids with large carbon numbers tend to be cracked in this reaction system. In some embodiments, stearic acid can be completely converted at 370° C. for 5 h and the selectivity to heptadecane was around 80%.

In some embodiments, the decarboxylation can be performed in an organic solvent including but not limited to acetonitrile.

In some embodiments, gas phase decarboxylation of hydrolyzed unsaturated fatty acids (e.g. short-chain, unsaturated free fatty acids) has been investigated in two fixed-bed reactors by changing reaction parameters such as temperatures, FFA feed rates, and H₂-to-FFA molar ratios. FFA, which contains mostly C₈ as well as a few C₆, C₁₀, and C₁₂ FFA, was fed into the boiling zone, evaporated, carried by hydrogen flow at the rate of 0.5-20 ml/min, and reacted with the 5% Pd/C catalyst in the reactor.

In some embodiments, single-stage continuous decarboxylation of straight-chain liquid hydrocarbons from free fatty acids is performed using one or more catalysts selected from the group of Mo on Al₂O₃, MgO on Al₂O₃, and Ni on Al₂O₃ and/or subcritical water. In some embodiments, straight-chain hydrocarbons were obtained via decarboxylation and hydrogenation reactions with no added hydrogen.

### Glycerolysis (Glycerol Esterification)

In one aspect, the method comprises combining glycerin and/or glycerol (e.g. glycerin and/or glycerol produced from acidification of the renewable oil) with unsaturated fatty acid (e.g. a portion of the short chain unsaturated fatty acid prepared from ethenolysis of unsaturated fatty acid (e.g. unsaturated free fatty acid)) to produce a mixture and subjecting the mixture to glycerolysis to produce short chain unsaturated acyl-glycerides.

In some embodiments, fatty acid esters (e.g. unsaturated fatty acid esters and saturated fatty acid esters) are converted into fatty acids (e.g. by saponification) which can then be decarboxylated. Non-limiting examples of methods for converting unsaturated fatty acid esters into unsaturated fatty acids and saturated fatty acid esters into saturated fatty acids includes treatment with aqueous alkali (e.g. NaOH).

Glycerol esterification or “glycerolysis” has been used to reduce FFA in low-grade oils without the use of acid, methanol or vacuum stripping. In some embodiments, when glycerin produced during ‘acid-hydrolysis’ and/or acification is combined with the short-chain, unsaturated free fatty acids at a temperature of approximately 238° C., the free fatty acids will react with glycerin to form an acyl glycerol or glyceride and water. In some embodiments, the resulting glycerides formed during glycerolysis can then be converted directly to biodiesel via base-catalyzed trans-esterification. In some embodiments, since glycerolysis is done at such high temperatures, any water formed is driven out immediately via a nitrogen purge. In some embodiments, the continuous removal of water throughout the process via a nitrogen purge is important for multiple reasons. In some embodiments, drying the renewable oil to moisture levels below 0.5% avoids the formation of excess soaps during base-catalyzed transesterification and the decanting problems that can occur. In some embodiments, purging the water from the system will also shift the reaction equilibrium toward the product side, allowing the free fatty acid concentration to fall below 0.2%. This is the result of Le Chatelier's principle, or “the equilibrium law”, which states that when a system at equilibrium is subjected to change (i.e., removal of water at low concentrations), the system will readjust itself to counteract the effect of the change, establishing a new equilibrium. In some embodiments, along with water, volatile organic compounds and/or light carboxylic acids are removed during the purge. In some embodiments, these compounds are the result of organic oxidation and can be very odorous. In some embodiments, blanketing the system with nitrogen avoids any further oxidation of the oil components at high temperature.

In some embodiments converting at least a portion of the free fatty acids in the acidified composition to acyl-glycerides includes esterification of the free fatty acids. As used herein, the term glycerolysis refers to the formation of acyl-glycerides by combining free fatty acids and glycerol in an inert environment.

In some embodiments, converting at least a portion of the free fatty acids in the acidified composition to glycerides includes contacting the free fatty acids in the acidified composition with glycerol.

In some embodiments, contacting the free fatty acids in the acidified composition with glycerol is conducted at a temperature from about 175° C.-260° C., 200° C.-255° C., 220° C.-250° C., 230° C.-245° C., or about 235° C.-240° C. or about 175° C., 200° C., 225° C., 230° C., 235° C., 238° C., 245° C., 250° C., or about 260° C.

In some embodiments, the converting at least the portion of the free fatty acids in the acidified composition to glycerides is acid catalyzed.

In some embodiments, the converting at least a portion of the free fatty acids in the acidified composition to glycerides is base catalyzed.

In some embodiments, the base catalyzed esterification, transesterification, and combinations thereof includes treating the free fatty acids with a methoxide, wherein the methoxide is chosen from sodium methoxide, potassium methoxide, lithium methoxide, zinc methoxide, calcium methoxide, tributyltin methoxide, magnesium methoxide, tantalum (V) methoxide, titanium (IV) methoxide, antimony (III) methoxide, germanium methoxide, copper (II) methoxide, and combinations thereof.

### Sustainable Lubricants

In one aspect, the present disclosure provides novel non-fossil, high performance sustainable lubricants that include non-fossil hydrocarbon molecule structures derived from sustainable plant biomass. In some embodiments, the lubricants comprise one or more base oils (e.g. hydrocarbon base oils). Non-limiting examples of base oils useful in the disclosure include long and short-chain alpha olefins, saturated hydrocarbons, and acyl-glycerides. In some embodiments, the lubricants of the disclosure outperform traditional high performance and synthetic petroleum products, are cost competitive with synthetic oils, have a direct drop-in compatibility with current systems, meet or exceed 19 applicable American Petroleum Institute (API) certifications, and/or are a viable alternative to inferior petroleum-based lubricants.

In some embodiments, the lubricants of the disclosure comprise base oils (e.g. hydrocarbon base oils) prepared using methods of the disclosure.

In some embodiments, the base oil comprises dimers, trimers, tetramers, and/or pentamers of C14-C18 olefin monomers (e.g. C14-C18 alpha olefin monomers). In some embodiments, the olefin monomer is a C16 olefin monomer (e.g. C16 alpha olefin monomer). In some embodiments, the base oil comprises C28-C36 dimers of C14-C18 olefin monomers (e.g. C14-C18 alpha olefin monomers), C42-C54 trimers of C14-C18 olefin monomers (e.g. C14-C18 alpha olefin monomers), C56-C72 tetramers of C14-C18 olefin monomers (e.g. C14-C18 alpha olefin monomers), and/or C70-C90 pentamers of C14-C18 olefin monomers (e.g. C14-C18 alpha olefin monomers). In some embodiments, the base oil comprises C32 dimers, C48 trimers, C64 tetramers, and/or C80 pentamers of C16 olefin monomers (e.g. C16 alpha olefin monomers). In one aspect, the present disclosure provides a lubricant comprising:

a) a saturated hydrocarbon base oil in an amount ranging from about 50 wt % to about 70 wt % of the total weight of the lubricant, wherein the saturated hydrocarbon base oil comprises oligomers of C14-C18 olefin monomers, the dimers having an average carbon number in a range of from 29 to 36;
b) a viscosity modifier in an amount ranging from about 1 wt % to about 30 wt % or about 20 wt % to about 30 wt % (e.g. about 1.4 wt %, about 1.80 wt %, about 3.2 wt %, about 4.13 wt %, about 5.2 wt %, or about 16.25 wt %, about 26 wt %) of the total weight of the lubricant;
c) a detergent in an amount ranging from about 10 wt % to about 15 wt % (e.g. about 12.3 wt %) of the total weight of the lubricant; and
d) a pour point depressant in an amount ranging from about 0.1 wt % to about 1 wt % (e.g. about 0.3 wt %) of the total weight of the lubricant.

In one aspect, the lubricant comprises a saturated hydrocarbon base oil. In some embodiments, the saturated hydrocarbon base oil comprises oligomers (e.g. dimers, trimers, tetramers, and/or pentamers) of C14-C18 olefin monomers. In some embodiments, the dimers have an average carbon number in a range of from 29 to 36. In some embodiments, the dimer portion has a weight average molecular weight in the range of from about 422 to about 510. In some embodiments, the trimers have an average carbon number in a range of from 42 to 55. In some embodiments, the tetramers have an average carbon number in a range of from 56 to 72. In some embodiments, the pentamers have an average carbon number in a range of from 70 to 90. Non-limiting examples of suitable saturated hydrocarbon base oils include SynNova 4 and SynNova 9. See also US 20200165538 and US 20200216772, each of which is incorporated by reference herein in its entirety.

In some embodiments, the lubricant of the disclosure comprises a viscosity index ranging from about 1% to about 30%, about 1% to about 6%, about 1% to about 17%, or about 205 to about 30% (e.g. about 1.4%, about 1.80%, about 3.2%, about 4.13%, about 5.2%, or about 16.25%, about 26%. In a non-limiting example, the lubricant is Euro OW40 and comprises and/or exhibits a viscosity index ranging from about 20% to about 30%, or about 25% to about 27%, or about 26%. In a non-limiting example, the lubricant is 5W30 and comprises and/or exhibits a viscosity index ranging from about 1% to about 6%, or about 3% to about 4%, or about 3.2%. In a non-limiting example, the lubricant is 5W20 and comprises and/or exhibits a viscosity index ranging from about 1% to about 6%, or about 1% to about 2%, or about 1.4%. In a non-limiting example, the lubricant is 5W40 and comprises and/or exhibits a viscosity index ranging from about 1% to about 6%, or about 4% to about 5%, or about 5.2%. In a non-limiting example, the lubricant is ISO 32 and comprises and/or exhibits a viscosity index ranging from about 1% to about 17%, or about 1% to about 2%, or about 1.80%. In a non-limiting example, the lubricant is ISO 46 and comprises and/or exhibits a viscosity index ranging from about 1% to about 17%, or about 4% to about 5%, or about 4.13%. In a non-limiting example, the lubricant is ISO 68 and comprises and/or exhibits a viscosity index ranging from about 1% to about 17%, or about 16% to about 17%, or about 16.25%.

In some embodiments, the lubricant comprises a saturated hydrocarbon base oil in an amount ranging from about 50 wt % to about 70 wt %, about 55 wt % to about 65 wt %, about 58 wt % to about 60 wt %, or about 59 wt % to about 60 wt % of the total weight of the lubricant. In some embodiments, the lubricant comprises a saturated hydrocarbon base oil in an amount of about 59 wt %, about 59.1 wt %, about 59.2 wt %, about 59.3 wt %, about 59.4 wt %, about 59.5 wt %, about 59.6 wt %, about 59.7 wt %, about 59.8 wt %, about 59.9 wt %, or about 60 wt % of the total weight of the lubricant.

In some embodiments, the saturated hydrocarbon base oil is a mixture or blend comprising two or more different saturated hydrocarbon base oils. In some embodiments, the saturated hydrocarbon base oil comprises two different saturated hydrocarbon base oils. In a non-limiting example, the saturated hydrocarbon base oil comprises SynNova 4 and SynNova 9. In some embodiments, the saturated hydrocarbon base oil comprises SynNova 4 in an amount ranging from about 50 wt % to about 60 wt %, about 52 wt % to about 58 wt %, about 55 wt % to about 57 wt %, or about 56 wt % to about 57 wt % of the total weight of the lubricant, and SynNova 9 in an amount ranging from about 3 wt % to about 7 wt %, about 4 wt % to about 6 wt %, or about 4.5 wt % to about 5.5 wt % of the total weight of the lubricant. In some embodiments, the saturated hydrocarbon base oil comprises SynNova 4 in an amount of about 56 wt %, about 56.1 wt %, about 56.2 wt %, about 56.3 wt %, about 56.4 wt %, about 56.5 wt %, about 56.6 wt %, about 56.7 wt %, about 56.8 wt %, about 56.9 wt %, or about 57 wt % of the total weight of the lubricant, and SynNova 9 in an amount ranging from about 4.5 wt %, about 4.6 wt %, about 4.7 wt %, about 4.8 wt %, about 4.9 wt %, about 5 wt %, about 5.1 wt %, about 5.2 wt %, about 5.3 wt %, about 5.4 wt %, or about 5.5 wt % of the total weight of the lubricant.

In one embodiment, the saturated hydrocarbon base oil comprises the dimers as a significant percent by weight of the base oil composition. In some embodiments, the saturated hydrocarbon base oil comprises the dimers in an amount of about 50 wt % or greater, about 80 wt % or greater, about 90 wt % or greater, about 95 wt % or greater, about 98 wt % or greater, or about 99 wt % or greater of the total weight of the lubricant.

In some embodiments, the saturated hydrocarbon base oil comprising the dimer is substantially absent of any 1-decene. For example, embodiments of the base oil may comprise less than 5% by weight of 1-decene in either monomer, dimer, or trimer form, as well as higher oligomer forms, such as less than 3% by weight of 1-decene, and even less than 1% by weight of 1-decene. In some embodiment, the saturated hydrocarbon base oil comprises less than about 10%, less than about 5%, or less than about 1% of dimers containing singularly branched isomers, according to the simulated distillation test ASTM D2887.

In some embodiments, the saturated hydrocarbon base oil, or each saturated hydrocarbon base oils when the saturated hydrocarbon base oil comprises two or more different saturated hydrocarbon base oils, exhibits one or more of the following properties:
a) a Noack Volatility as measured by ASTM D5800 and/or CEC L-40-A-93 that is less than about 14%, less than about 13%, less than about 12%, less than about 11%, less than about 10%, or less than about 9% (e.g. about 7.4%);
b) a Bromine Index below about 1000 mg Br2/100 g, about 500 mg Br2/100 g, or below about 200 mg Br2/100 g as determined in accordance with D2710-09;
c) an average branching index (BI) as determined by 1H NMR that is in the range of about 22 to about 26;
d) an average paraffin branching proximity (BP) as determined by 13C NMR in a range of from about 18 to about 26;
e) a viscosity index as determined in accordance with ASTM D2270 of about 125 or greater, about 130 or greater, about 135 or greater, or about 140 or greater;
f) a pour point as determined in accordance with ASTM D97 less than about −20° C., less than about −27° C., less than about −30° C., less than about −33° C., less than about −36° C., less than about −39° C., or less than about −42° C.;
g) a Cold Crank Simulated (CCS) dynamic viscosity as measured by ASTM D5293 at −35° C. less than about 1800 cP, less than about 1700 cP, less than about 1600 cP, less than about 1500 cP, less than about 1400 cP, less than about 1300 cP, less than about 1200 cP, or less than about 1100 cP; and/or
h) a KV (100) as measured by ASTM D445-17a that is in the range of about 3.7 cSt to about 9.7 cSt, or about 3.7 cSt to about 4.8 cSt.

The average paraffin branching proximity (BP) is a measure of the content of recurring methylene groups in the dimer portion according to the following formula:
paraffin branching proximity (BP)=(number of ε carbon groups/total number of carbon groups)*100;
where an ε carbon group is defined as α carbon group that is separated from any terminal carbon atom groups or branching carbon groups by at least 4 carbon group.

The branching index is a measure of the extent of branching, and can be determined according to the following formula:

Branching index (BI)-(total content of methyl group hydrogens/total content of hydrogens)*100.

In one aspect, the lubricant comprises a viscosity modifier. In some embodiments, viscosity modifiers are used to minimize lubricant viscosity at lower temperatures, meet industry performance standards, have excellent shear stability at a low treat rate, retain low temperature performance and provide viscosity control at high temperatures. Any viscosity modifier is contemplated by the present disclosure, as would be understood by one of ordinary skill in the art. Non-limiting examples of viscosity modifiers include Infineum SV603 and Infineum SV261L.

In some embodiments, the lubricant comprises a viscosity modifier in an amount ranging from about 20 wt % to about 30 wt %, about 21 wt % to about 29 wt %, about 22 wt % to about 28 wt %, about 23 wt % to about 27 wt %, about 25 wt % to about 27 wt %, or about 25.5 wt % to about 26.5 wt % of the total weight of the lubricant. In some embodiments, the lubricant comprises a viscosity modifier in an amount of about 25 wt %, about 25.1 wt %, about 25.2 wt %, about 25.3 wt %, about 25.4 wt %, about 25.5 wt %, about 25.6 wt %, about 25.7 wt %, about 25.8 wt %, about 25.9 wt %, about 26 wt %, about 26.1 wt %, about 26.2 wt %, about 26.3 wt %, about 26.4 wt %, about 26.5 wt %, about 26.6 wt %, about 26.7 wt %, about 26.8 wt %, about 26.9 wt %, or about 27 wt % of the total weight of the lubricant.

In one aspect, the lubricant comprises a detergent. In some embodiments, detergents are used to neutralize acidic blow-by gases, control rust, reduce lacquer and prevent deposits on engine components such as pistons. Any detergent is contemplated by the present disclosure, as would be understood by one of ordinary skill in the art. Non-limiting examples of detergents include Infineum P6003.

In some embodiments, the lubricant comprises a detergent in an amount ranging from about 10 wt % to about 15 wt %, about 11 wt % to about 14 wt %, or about 12 wt % to about 13 wt % of the total weight of the lubricant. In some embodiments, the lubricant comprises a detergent in an amount of about 12 wt %, about 12.1 wt %, about 12.2 wt %, about 12.3 wt %, about 12.4 wt %, about 12.5 wt %, about 12.6 wt %, about 12.7 wt %, about 12.8 wt %, about 12.9 wt %, or about 13 wt % of the total weight of the lubricant.

In one aspect, the lubricant comprises a pour point depressant. In some embodiments, pour point depressants are used to prevent wax crystals in lubricants from agglomerating or fusing together at reduced ambient temperatures. In some embodiments, the pour point depressant is also useful as a flow improver. Any pour point depressant is contemplated by the present disclosure, as would be understood by one of ordinary skill in the art. Non-limiting examples of pour point depressants include Infineum V385.

In some embodiments, the lubricant comprises a pour point depressant in an amount ranging from about 0.1 wt % to about 1 wt %, 0.1 wt % to about 0.5 wt %, or 0.2 wt % to about 0.4 wt % of the total weight of the lubricant. In some embodiments, the lubricant comprises a pour point depressant in an amount of about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, or about 1 wt % of the total weight of the lubricant.

In some embodiments, the lubricant further comprises one or more additives. Non-limiting examples of additives include anti-wear additives. In some embodiments, anti-wear additives comprise zinc dialkyl dithiophosphate (ZDDP). Non-limiting examples of additives comprising ZDDP include Infineum D3337, Infineum P5920, and Infineum P6003.

In one embodiment, the lubricant comprises:
a) a saturated hydrocarbon base oil comprising SynNova 4 in an amount ranging from about 56 wt % to about 57 wt % of the total weight of the lubricant, and SynNova 9 in an amount ranging from about 4.5 wt % to about 5.5 wt % of the total weight of the lubricant;
b) a viscosity modifier comprising Infineum SV603 in an amount ranging from about 25.5 wt % to about 26.5 wt % of the total weight of the lubricant;
c) a detergent comprising Infineum P6003 in an amount ranging from about 12 wt % to about 13 wt % of the total weight of the lubricant; and
d) a pour point depressant comprising Infineum V385 in an amount ranging from about 0.2 wt % to about 0.4 wt % of the total weight of the lubricant.

In one embodiment, the lubricant comprises:
a) SynNova 4 in an amount of about 56.4 wt % of the total weight of the lubricant;
b) SynNova 9 in an amount of about 5 wt % of the total weight of the lubricant;
c) Infineum SV603 in an amount of about 26 wt % of the total weight of the lubricant;
d) Infineum P6003 in an amount of about 12.3 wt % of the total weight of the lubricant; and
e) Infineum V385 in an amount about 0.3 wt % of the total weight of the lubricant.

While preferred embodiments of the disclosure are shown and described herein, such embodiments are provided by way of example only and are not intended to otherwise limit the scope of the disclosure, and various alternatives to the described embodiments of the disclosure may be employed in practice.

### Examples

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Exemplary Preparation of Renewable Oil Base Stock

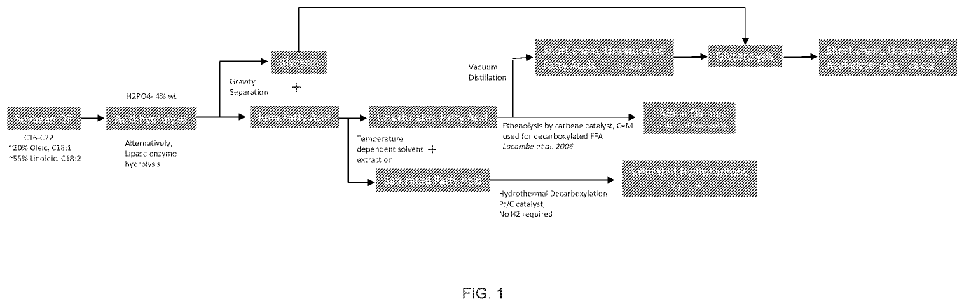

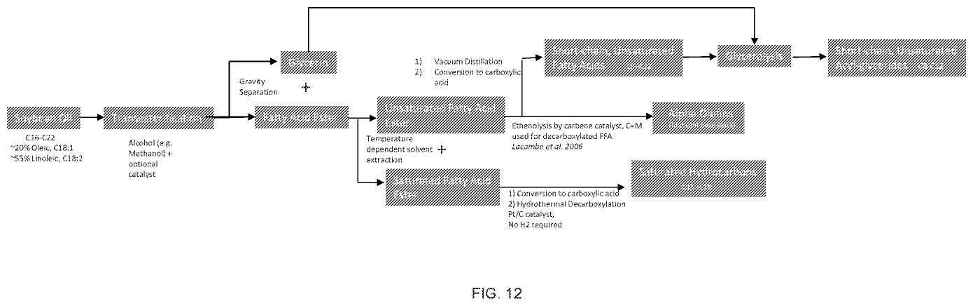

Non-limiting examples of methods for preparing base oils/base stocks from renewable oils are shown in FIGS. 1 and 12.

### Oil Pretreatment

The first step is to pretreat approx. 7.8 L (2 gal) of the chosen renewable oil precursor, in this case soybean oil. The initial size of the pretreated oil is dependent upon multiple factors; including but not limited to unforeseen sample loss, multiple reaction scenarios requiring assay test development, and lab-scale equipment limitations to cover all unit operations.

Using commercially available filtration aids (diatomaceous earth, cellulosic filter aid, filter bags at a combination of filter pore sizes), an oil filtration method can be established. The purpose of this stage is to produce an oil precursor of high enough quality to continue through the lubricant process, not to optimize the filter-aid to oil ratio. The final material should be free of debris and unable to be separated via lab centrifugation.

Oil Pretreatment

| No. | Chemicals and Equipment |
|---|---|
| 1 | Renewable Oil |
| 2 | 5 L Round bottom flask |
| 3 | Filter Media (diatemaceous eart, activated carbon, cellulosic, |
| | filter paper) |
| 4 | Filter apparatus w/applicable glassware |
| 5 | Solvent vacuum pump |
| 6 | 4-point decimal analytical balance |
| 7 | Course mass balance for bulk tare weights |
| | Analytical Analysis Performed (x3) |
| 8 | Karl Fisher Titrator for moisture analysis |
| 9 | GC-mass spec for qualitative analysis of incoming oil profile |
| 10 | GC-FID for quantitative analysis of incoming oil profile |
| 11 | Muffle furnace for Prox. Analysis of incoming oil |
| 12 | pH probe |

### Acid Hydrolysis of Renewable Oil

Collecting the filtered oil from the pretreatment stage, two, single-stage acid-hydrolysis reactions are performed on approx. 7.5 L of oil. In a non-limiting embodiment, the reaction is carried out between fats or oils at a suitable temperature (˜100 C, refluxing or 120° C. under pressure) and with provision for mixing either by steam or by mechanical stirring. In addition, small quantities of mineral acids (1% sulfuric acid wt/wt relative to oil) or organic sulfonic acids known as “saponifiers’ “emulsifiers”, “splitting agents”, etc. Using two acid types, sulfuric acid (97% wt/wt) and methyl sulfonic acid (MSA), allows for some degree of performance optimization. The result of the test comparison has significant impact on the overall operational cost of any developed facilities.

In process, the oil is heated and agitated to the desired temperature in the presence of stoichiometric excess of water (4 X). Once to temperature, the chosen acid is charged to either a refluxing vessel at atmospheric pressure and/or to an autoclave reactor, under positive pressure and elevated temperature (120° C.). The reaction mixture is vigorously agitated for 1 hour before stopping the reaction and transferring to a separatory funnel. After phase separation, the upper free fatty acid layer will be tested, and the rate of reaction established. Depending on the analytical results, the free fatty acid phase layer can either be re-hydrolyzed of sent on to the next stage. All weights, data, and reaction parameters are recorded for later analysis.

Acid Hydrolysis of Renewable Oil (Twitchell Splitting)

| No. | Chemicals and Equipment |
|---|---|
| 1 | Filtered and/or degummed oil |
| 2 | 5 L Round bottom flask |
| 3 | 5 L flask glass agitator |
| 4 | 5 L flask heating mantle w/top glass insulation |
| 5 | Reflux Condenser w/chilled glycol circulating bath |
| 6 | Catalyst (metal impregnated ceramic) |
| 7 | Sulfonic Acid (MSA and/or Sulfuric Acid (97% wt/wt)) |
| 8 | 3 L Separatory Funnel (2) |
| 9 | 4-point decimal analytical balance |
| 10 | Course mass balance for bulk tare weights |
| | Analytical Analysis Performed (x3) |
| 11 | Karl Fisher Titrator for moisture analysis |
| 12 | GC-mass spec for qualitative analysis of FFA profile |
| 13 | GC-FID for quantitative analysis of FFA profile |
| 14 | Muffle furnace for Prox. Analysis of FFA and Glycerol |
| 15 | pH probe for initial and final readings for FFA and Glycerol |
| 16 | Conductivity assay of glycerin separation |

### Chemical Separation of Free Fatty Acids

After the separation free fatty acids from glycerol, the upper phase is transferred to a 5 L round-bottom reactor with agitation. In 2 L volumes, the two hydrolysis trials are subjected to an acetone extraction reaction. Using a 1:1 ratio of oil to solvent, the mixture is agitated for 20 min at 60° C., under chilled reflux using ethylene glycerol and water (50% wt/wt) as the condenser media. Once the reaction is complete, the reactor contents are transferred to 4 individual 1.0 L close-lid beakers to be chilled to 0° C., in ethylene glycol and water baths. To guarantee complete saturation, the beakers are chilled for 24 hours before separating the solid, saturated free fatty acids from the liquid, unsaturated free fatty acids. In a non-limiting embodiment, only the liquid portion is of interest to produce the lubricant base stock. The saturated portion of the free fatty acids is analyzed, and its character determined for the purpose of providing a useful substrate for renewable diesel production (i.e., iodine value, % inert content, FFA profile, etc.). The liquid, unsaturated portion is placed transferred to a rotor evaporator, to evaporate the bulk of the acetone solvent at low temperatures. After the bulk of the solvent is removed, the beakers containing the extracted unsaturated FFAs are placed in a vacuum chamber to removed trace solvents before moving on to the next unit operation.

Chemical Separation of Unsaturated and Saturated Free Fatty Acids

| No. | Chemicals and Equipment |
|---|---|
| 1 | Free Fatty Acid (FFA) phase |
| 2 | 5 L Round bottom flask |
| 3 | 5 L flask glass agitator |
| 4 | 5 L flask heating mantle w/top glass insulation |
| 5 | Reflux Condenser w/chilled glycol circulating bath |
| 6 | Chilled glycol bath, temp set to 32 F. |
| 7 | 3 L Separatory Funnel (2) |
| 8 | ACS grade Acetone (4 L) |
| 9 | Filter apparatus w/applicable glasswear |
| 10 | Solvent vacuum pump |
| 11 | Rotary evaporator for solvent recovery |
| 12 | 4-point decimal analytical balance |
| 13 | Course mass balance for bulk tare weights |
| | Analytical Analysis Performed (x3) |
| 14 | Karl Fisher Titrator for moisture analysis |
| 15 | GC-mass spec for qualitative analysis for sat. and unsat. FFA |
| | profile |
| 16 | GC-FID for quantitative analysis of sat. and unsat. FFA profile |
| 17 | Muffle funace for Prox. Analysis of sat. and unsat. FFA |
| 18 | pH probe |

### Ethenolysis of Unsaturated Free Fatty Acids

Using the unsaturated free fatty acids purified during the last stage, approx. 0.5 L of material is charged to a 1 L Parr reactor (series 4843 w/temp control and mixing). In conjunction with the oil, there is a non-ionic solvent (dichloro-methane or n-hexane), heterogeneous catalyst (multiple types cited below), and gaseous ethylene are combined in the reactor at previously published charges based on previous research. The reaction conditions are held at approx. 40° C. and 10 bar of pressure for over 10 hrs. In a non-limiting example, the results recreate published results to obtain a lubricating base stock with the desired specifications.

The reaction is deemed complete once the product components are quantitatively determined and meet the published yield. Once this has been established, the oil phase is separated via rotor evaporation from the gaseous and solvent components. No significant solvent of gas recovery systems is being implemented at this stage due to the scale of the research. After the bulk of the solvent is removed via atmospheric evaporation, the beakers containing the extracted alpha olefins and newly formed short-chain free fatty acids are placed in a vacuum chamber to removed trace solvents before moving on to the next unit operation.

Ethenolysis of Unsaturated Free Fatty Acids

| No. | Chemicals and Equipment |
|---|---|
| 1 | Unsaturated Free Fatty Acid (FFA) phase |
| 2 | Parr HP/HT reactor, series 4843 |
| 3 | Gaseous ethylene (linde, 99.0%) |
| 4 | Mo—Al2O3 - catalyst #1 |
| 5 | Ni—Al2O3 - catalyst #2 |
| 6 | Ni/C impregnated ceramic catalyst - catalyst #3 |
| 7 | Non-ionic liquid (DCM or n-hexane) |
| 8 | Dry vacuum drum, removes residual ethylene |
| 9 | 4-point decimal analytical balance |
| 10 | Course mass balance for bulk tare weights |
| 11 | Multiple gas pressure regulators to step pressure up and down |
| | Analytical Analysis Performed (x3) |
| 12 | Karl Fisher Titrator for moisture analysis |
| 13 | GC-mass spec for qualitative analysis of alpha olefin profile |
| 14 | GC-FID for quantitative analysis of alpha olefin profile |
| 15 | GC-mass spec for qualitative analysis of short-chain FFA |
| 16 | GC-FID for quantitative analysis of short-chain FFA |
| 17 | pH probe |

### Hydroisomerization of Alpha Olefins to Lubricant Base Stock

To convert the alpha olefin portion of the resulting mixture to a desired form, it first needed to be separated from short-chain FFA. This can be achieved using several different extraction techniques, including but not limited to a co-solvent extraction to fractional distillation. Depending on the characteristic of the resulting laboratory mixture, a separation approach is determined based-on need. Although not wishing to be limited by theory, differences in polarity and specific gravity between the two majority components (alpha olefins and short-chain FFAs) may result in an innate phase separation, avoiding the need for additional chemical treatments.

After separating the alpha olefin portion, it oligomerized in the presence of a heterogenous catalyst (AlCl₃ and BF₃). The reaction is carried out in a Parr reactor (series 4843 w/continuous mixing). Post oligomerization analysis is required to confirm the degree of reaction. Once the oligomerization (e.g. dimerization) reaches published levels, the material is ready to be isomerized either in the presence of hydrogen or under inert conditions. The purpose of isomerization is to increase the terminal branching character to the oligomerized (e.g. dimerized) alpha olefin, while the hydrogen is used to saturate any residual double-bond character. Isomerization and hydrogenation can be performed inside the parr reactor, with the degree of isomerization being controlled by the heterogenous catalyst type, temperature, pressure, and residence time. Reaction conditions for isomerization reactions can range above 200 C and 2,000 psi, in the presence of heterogenous catalysts like AlCl₃ and BF₃. Catalyst loading, temperatures, and pressure parameters are published and can be developed for lab applications.

Oligomerization and Hydroisomerization
of Alpha Olefins to Lubricant Basestock

| No. | Chemicals and Equipment |
|---|---|
| 1 | Alpha Olefin mixture |
| 2 | Parr HP/HT reactor, series 4843 |
| 3 | Hydrogen gas (47raxair, 99.95%) |
| 4 | ALCL3 and/or BF3 complexes as catalyst |
| 5 | Dry vacuum drum, removes residual ethylene |
| 6 | Fractional distillation column, lab-scale |
| 7 | 4-point decimal analytical balance |
| 8 | Course mass balance for bulk tare weights |
| 9 | Multiple pressure regulators to step pressure up and down |
| | Analytical Analysis Performed (x3) |
| 10 | Karl Fisher Titrator for moisture analysis |
| 11 | GC-mass spec for qualitative analysis of alpha olefin profile |
| 12 | GC-FID for quantitative analysis of alpha olefin profile |
| 13 | pH probe |

### Example 2: Lubricant 0W-40

The lubricant OW-40 comprising the following components shown in Table 1 was prepared:

TABLE 1
Components of Lubricant 0W-40

| Component | % by weight |
|---|---|
| Infineum P6003 | 12.3 |
| Infineum SV603 | 26.0 |
| Infineum V385 | 0.3 |
| SynNova 4 | 56.4 |
| SynNova 9 | 5.0 |

Analyses were performed in accordance with the ASTM test procedures used with no deviations or modifications. Precision for these test results should be consistent with that stated in the test procedures referenced. Test results are shown in Tables 2 and 3 below.

TABLE 2
ASTM Tests Performed on Lubricant 0W-40

| Test | 0W-40 |
|---|---|
| ASTM D2896 Base Number- Procedure A; mg KOH/g | 7.72 |
| ASTM D4683 Apparent Viscosity at 150° C., cP | 3.87 |
| ASTM D4684 Borderline Pumping Temperature Test | |
| Apparent Viscosity @ −40° C., cP | 18,600 |
| Yield Stress, Pa | <35 |
| ASTM D6186 Oxidation Induction @ 210° C., min. | 30 |
| ASTM D7109 Shear Stability (90 Cycles) | See Table |
| | 3 below |

TABLE 3
ASTM D7109 Shear Stability

| | |
|---|---|
| New Oil Viscosity @ 100° C., mm²/s | 13.91 |
| Sheared Oil (30 Cycles) Viscosity @ 100° C., mm²/s | 13.73 |
| Viscosity Loss (30 Cycles), % | 1.29 |
| Sheared Oil (90 Cycles) Viscosity @ 100° C., mm²/s | 13.63 |
| Viscosity Loss (90 Cycles), % | 2.01 |
| Calibration Pressure, MPa | 12.7 |

### Example 3: Oil Purification-Filtration and Degumming

### Materials

2-L soybean oil (Purchased Oleic Acid (Carolina Chemical) TAN(D974)), 5 L Round bottom flask, separatory funnel, citric acid, potable water, filter media (diatomaceous earth), filter paper (50-micron) and filter apparatus with applicable glassware.

### Method

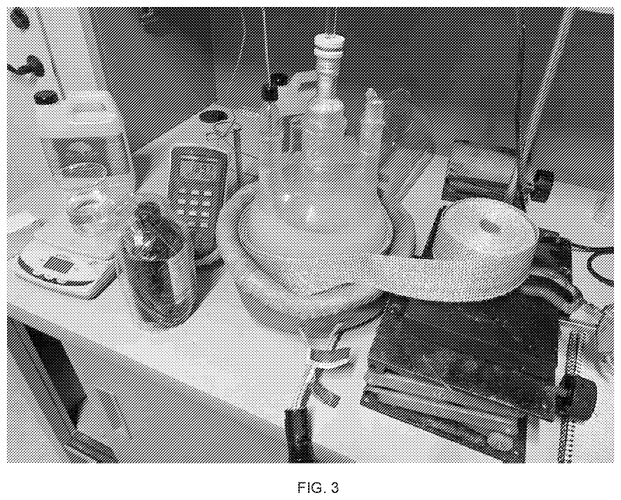

Refined soybean oil was purchased and degummed, as shown in FIG. 3, to ensure tri-glyceride purity. Two-thousand grams of soybean oil was mixed with 400 g (20% wt/wt) water and citric acid solution (5% citric acid in water, wt/wt) at 150° F. for 30 min. After the reaction the mixture settled for 1 hr in a separatory funnel before being decanted. The upper oil phase was strained through a filter media (diatomaceous earth) and filter paper (50-microns) at 120° F. Recovered 1995 g of soybean oil post-filtration. A 20 g sample was sent for third-party analytical analysis, for free fatty acid profile analysis.

### Acid Hydrolysis of Feed Oil (Twitchell Splitting)

### Trial #1-Atmospheric Reflux

### Materials

2-L degummed soybean oil, 5 L Round bottom flask, 5 L flask glass agitator, 5 L flask heating mantle w/top glass insulation, Reflux Condenser w/chilled glycol circulating bath, Sulfuric Acid (97% wt %), separatory funnel.

### Method #2

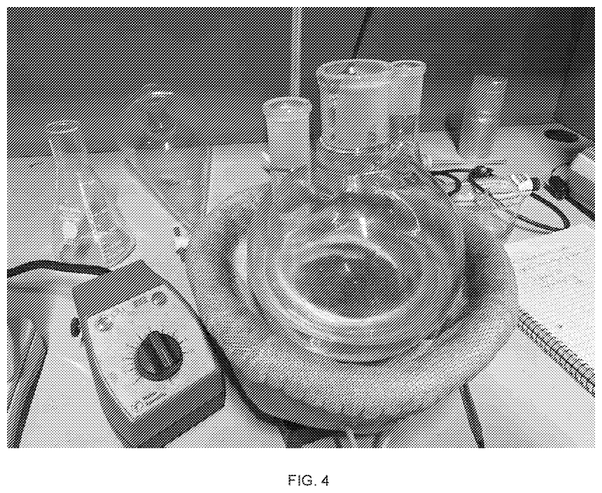

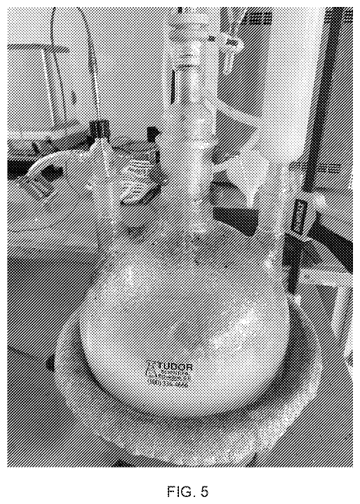

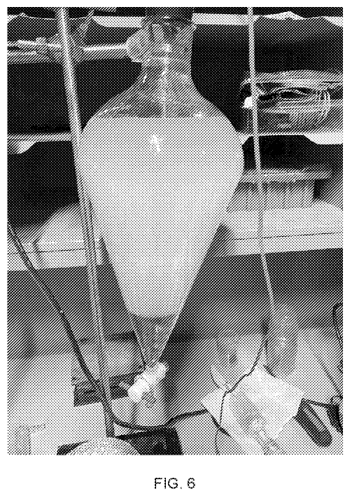

Refined soybean oil (1653 g), as shown in FIG. 4, was heated in a 5 L round bottom flash to 150 degrees F. The calculated stoichiometric equivalence of water for hydrolysis equaled 34.5 g water, but the actual amount charged was 10×stoichiometric (345 g). Sulfuric acid (98%) was added to the mixture at 4% wt/wt, relative to soybean oil (66 g sulfuric acid). The mixture was refluxed at 235° F. in a 5 L round bottom flash for 12 hrs. The condenser temperature was maintained at 15° F., guaranteeing all water vapor was returned to the reactor. The acid hydrolysis is shown in process in FIG. 5. After the reaction was complete, the mixture was allowed to cool to room temperature before being charged to a separatory funnel. The lower water/glycerin phase (460 g) was removed after 60 min of settling. The upper oil phase (1630 g) was removed, weighed, and sampled for analytical testing. These layers of oil and water/glycerin are illustrated in FIG. 6. Subsequent testing showed that the triglyceride to free fatty acid conversion only yielded 20% at lower temperatures.

### Trial #2-High Pressure/High Temperature Reactor

### Materials

2-L degummed soybean oil, 1-L high-temperature, high-pressure, stainless-steel mix reactor, nitrogen gas, separatory funnel.

### Method #2

In response to Trial #1, the reaction conditions for acid hydrolysis were modified to achieve a higher reaction temperature. Refined soybean oil (500 g) is to be mixed with water (250 g) prior to being charged into a high-temperature, high-pressure mixed reactor. The vapor space of the reactor is purged with nitrogen gas, to avoid unwanted oxidation. The reactor is heated to 500° F. while the internal pressure rises to 870 psi. The reaction conditions are maintained for 3 hrs with agitation. After completion, the reactor is cooled to room temperature and excess pressure released. The reactor contents are allowed to settle in a separatory funnel for 2 hrs, allowing the dense glycerin and excess water phase to collect and be removed via a separatory funnel. The glycerin is vacuum dried at 240° F. and 60 mmHg while maintaining conditions for 20 min. The lower density free fatty acid component are dried at the same conditions as the glycerin and are expected to yield approx. 89% wt/wt free fatty acids

### Chemical Separation of Unsaturated and Saturated Free Fatty Acids

### Materials

Free Fatty Acid (FFA) phase, double-jacketed, glass-lined reactor, 5 L flask glass agitator, chilled ethylene glycol bath, separatory funnel, ACS grade acetone, filter apparatus with applicable glassware, solvent vacuum pump, rotary evaporator for solvent recovery.

### Method

Free fatty acids (1630 g) produced during acid-hydrolysis (trial #1) were combined with ACS grade acetone (1:1.5, oil to solvent). The two components were mixed before being chilled to 0° F. for 24 hrs using a chilled circulation bath. After sub-cooling the mixture, the contents were filtered immediately upon removal from the chilled system. The material was filtered through a chilled porous cloth that could retain the solid phase, crystalized free fatty acids. The liquid filtrate was weighed before the residual acetone was evaporated using a rotary evaporator under vacuum (100 mmHg). The resulting oil (8.2% wt/wt) was sampled and tested to confirm degree of unsaturation and free fatty acid profile. Test results from acid-hydrolysis (trial #1) indicated the lack of free fatty acid conversion was cause for the low extraction yield. Repeating the acid-hydrolysis process using trial #2 method is expected to improve the yield during extraction.

### Example 4: Decarboxylation and Ethenolysis

Fatty acids are decarboxylated using silver (+2) as a catalyst which is generated from silver (+1) by the action of sodium persulfate. The reaction is conducted in an acetonitrile+water solvent system at the reflux temperature (˜78° C.) with a 20 minute reaction time.

### Oxidative Decarboxylation of Oleic Acid

### Reactants:

1.9874 gm of Oleic acid (Carolina Biological Supply Co.-lab grade cat #87-8340)
1.31 gm of silver nitrate (MCB-reagent grade cat #SX205) diluted in 50 mls DI water.
90 mls of acetonitrile (EMD-HPLC grade >99.99% cat #AX0142-1)
3.70 gms of Sodium persulfate (Sigma-Aldrich->98% cat #216232) diluted in 40 mls DI water

### Apparatus:

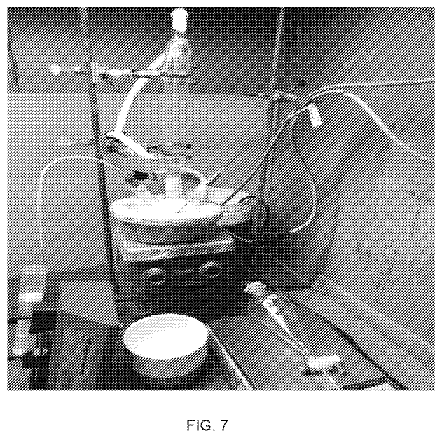

250 cc three-neck boiling flask fitted with a water cooled condenser, SS type K TC, a ⅛″
Teflon liquid addition tube, and magnetic stir bar. A heating mantle and power control is used to adjust the reaction mix temperature. The boiling flask and heating mantle is placed on a magnetic stirrer for constant agitation during the experiment. A constant speed syringe pump is used to deliver the sodium persulfate solution from a 50 cc syringe into the reaction mixture. An example of this experimental setup is shown in FIG. 7.

### Procedure:

The oleic acid, silver nitrate solution, and acetonitrile are placed in the boiling flask. The cooling water for the condenser is started and the heating mantle is powered up. After the reaction mixture has reached ˜78° C. some boiling action and/or refluxing at the base of the condenser should be visible. At this time the slow addition (4 mls/min) of the sodium persulfate is started. After all of the persulfate solution has been added (10 min), the reaction is continued for an additional 10 minutes. At the end of the reaction period, the reaction mixture is quickly cooled to room temperature by removing the heating mantle and replacing it with an ice bath.

### Product Recovery:

After cooling add 0.877 gms of nC16 to the reaction mixture and mix well. Transfer the reaction mixture to a 250 cc separatory funnel and wash three times with ethyl ether. Wash out the 250 cc reaction flask with each ethyl ether wash and add to reaction mixture. Combine the three ethyl ether extracts and wash twice with saturated sodium bicarbonate solution.

### Analysis:

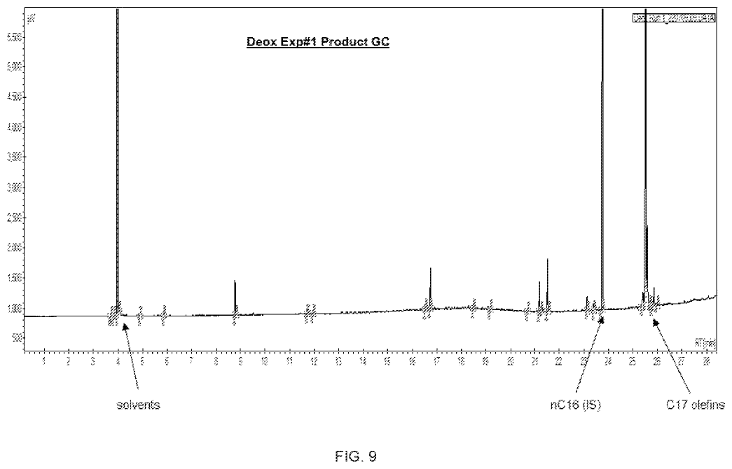

Filter 2 mls of final ethyl ether extract (0.45 um) into a 2 ml vial and analyze by GC. GC setup:
Column-100Mx0.25 mmx1 um RTx-5
Temp Prog−100° C.-2 min-6 deg/min−300° C. final-20 min
Injection-1 ul split 100:1 at 300° C.
Carrier gas-Hydrogen @46 psig
Detector-FID @300° C.
   Results: C17 mono-olefins (ID by MS) yield=˜32 wt % as shown in FIG. 9.

### Ethenolysis of Fatty Acids, Esters or Internal Olefins

Olefin metathesis with ethylene and unsaturated fatty esters or internal olefins result in a mixture of shorter chain alpha-olefins and methyl esters. In a non-limiting example, ionic liquids can be used as a reaction solvent, which allows for the multiple reuse of the expensive Grubbs catalyst. This Example demonstrates the use of lower cost solvents than ionic liquids for ethenolysis of fatty esters initially and fatty acids as feedstock.

### Experiment #1-Ethenolysis of Methyl Oleate (FAME)

### Reactants:

0.0425 gms of HG2 (Grubbs (II)) catalyst
1.0544 gms of methyl oleate
20 mls of dry Isooctane-dried over 5A MS
Ethylene gas at >100 psig->99% purity

### Apparatus:

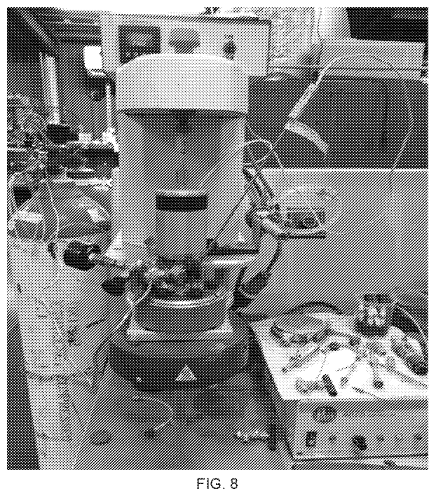

160 cc Parr SS autoclave was fitted with a glass liner to reduce reaction volume to about 50 cc. The reactor is fitted with SS type K TC, mag drive internal stirrer, multiple ports for N2 purge, ethylene addition, pressure sensing, and syringe injection of dry solvent under N₂ purge. Lower reactor section can be heated with a removable external heater. An example of this experimental setup is shown in FIG. 8.

### Procedure:

Prior to experiment, a hot (225-250° F.) N₂ purge of the liner and autoclave is performed overnight and allowed to completely cool. Quickly open the autoclave and remove glass liner and place in a N2 purged desicator. Reassemble autoclave partially and maintain dry N2 purge until ready to insert charged liner. Set up a N2 purge in the draft cover on the analytical balance for about 30 minutes prior to weighing reactants. Working quickly, weigh out the required HG2 catalyst and liquid feed using the liquid feed to cover the HG2 catalyst powder to prevent oxidation. Transport the charged liner back to the autoclave in the N2 purged desicator. Again, working quickly, briefly open the autoclave and reinstall the charged liner, close the autoclave and re-establish the dry N2 purge while tightening the autoclave closure to leak tight condition. Leak check autoclave with dry N2 to 100 psig. Continue dry N2 purge of HG2 catalyst and feed for one hour before introducing the dry isooctane to reactor while N2 purging. Continue a slow N2 purge for 10 minutes before switching the gas flow to ethylene and performing 3-4 pressure-depressure cycles quickly at 20-30 psig before finally pressurizing the system to 100 psig with ethylene. Continue to monitor the ethylene pressure and reaction temperature until ethylene pressure stabilizes in the reactor. Reaction time was 8 hours, and temperature varied from 29-36° C.

### Product Recovery:

Allow the reactor to slowly depressurize and replace ethylene with dry nitrogen to inert the reactor prior to opening. Pipet the reactor contents into a small bottle with three small flushes of dry isooctane. Add a few drops of water to the bottle and shake well to deactivate the catalyst. Add 0.4954 gms of nC14 (IS) and mix well.

### Analysis:

Filter 2 mls of reaction product (0.45 um) into a 2 ml vial and analyze by GC.

### GC Setup:

Column-100Mx0.25 mmx1 um RTx-5
Temp Prog−100° C.-2 min-6 deg/min−300° C. final-20 min
Injection-1 ul split 100:1 at 300° C.
Carrier gas-Hydrogen @46 psig
Detector-FID @300 C

### Results:

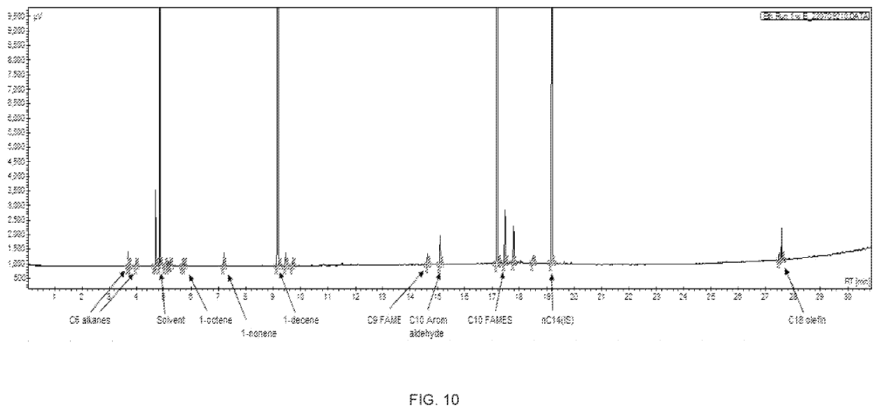

˜41 wt % 1-decene yield (based on methyl oleate wt). Methyl oleate conversion ˜99 wt %. FIG. 10 illustrates the chromatogram illustrating these results.

### Experiment #2-Ethenolysis of Oleic Acid

### Reactants:

0.0450 gms of HG2 catalyst
0.9986 gms of oleic acid
20 mls of dry Isooctane-dried over 5A MS
Ethylene gas at >100 psig→99% purity

### Apparatus:

160 cc Parr SS autoclave fitted with a glass liner to reduce reaction volume to about 50 cc. The reactor is fitted with SS type K TC, mag drive internal stirrer, multiple ports for N2 purge, ethylene addition, pressure sensing, and syringe injection of dry solvent under N₂ purge. Lower reactor section can be heated with a removable external heater. An example of this experimental setup is shown in FIG. 8.

### Procedure:

Prior to experiment perform a hot (225-250° F.) N2 purge of the liner and autoclave overnight and allow to completely cool. Quickly open the autoclave and remove glass liner and place in a N2 purged desicator. Reassemble autoclave partially and maintain dry N2 purge until ready to insert charged liner. Set up a N2 purge in the draft cover on the analytical balance for about 30 minutes prior to weighing reactants. Working quickly, weigh out the required HG2 catalyst and liquid feed using the liquid feed to cover the HG2 catalyst powder to prevent oxidation. Transport the charged liner back to the autoclave in the N2 purged desicator. Again, working quickly, briefly open the autoclave and reinstall the charged liner, close the autoclave and re-establish the dry N2 purge while tightening the autoclave closure to leak tight condition. Leak check autoclave with dry N2 to 100 psig. Continue dry N2 purge of HG2 catalyst and feed for one hour before introducing the dry isooctane to reactor while N2 purging. Continue a slow N2 purge for 10 minutes before switching the gas flow to ethylene and performing 3-4 pressure-depressure cycles quickly at 20-30 psig before finally pressurizing the system to 100 psig with ethylene. Continue to monitor the ethylene pressure and reaction temperature until ethylene pressure stabilizes in the reactor. Reaction time was 8 hours, and temperature varied from 29-36° C.

### Product Recovery:

Allow the reactor to slowly depressurize and replace ethylene with dry nitrogen to inert the reactor prior to opening. Pipet the reactor contents into a small bottle with three small flushes of dry isooctane. Add a few drops of water to the bottle and shake well to deactivate the catalyst. Add 0.4954 gms of nC14 (IS) and mix well.

### Analysis:

Filter 2 mls of reaction product (0.45 um) into a 2 ml vial and analyze by GC.

GC setup:
Column-100Mx0.25 mmx1 um RTx-5
Temp Prog−100° C.-2 min-6 deg/min−300° C. final-20 min
Injection-1 ul split 100:1 at 300° C.
Carrier gas-Hydrogen @46 psig.
Detector-FID @300 C

### Results:

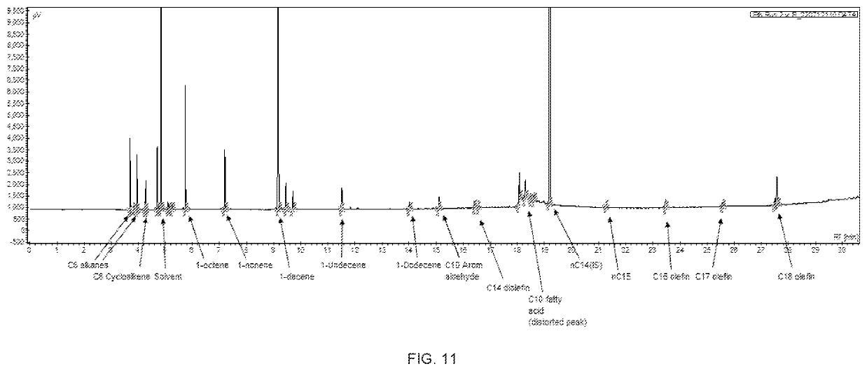

˜22% 1-decene yield, approximately 10% of other compounds not observed in experiment #1 or at much higher levels. Not able to measure residual oleic acid so conversion could not be calculated. Refer to FIG. 11 illustrating these results.

### Example 5: ANALYSIS OF BASE OIL

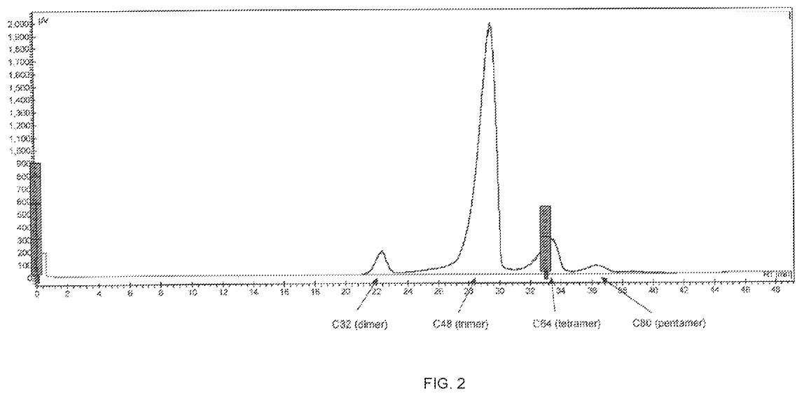

Base oil sample was analyzed by gas chromatography. The base oil was found to include C32 dimer), C48 (trimer), C64 (tetramer), and C80 (pentamer). FIG. 2 illustrates the gas chromatography trace. Carbon assignments are based on oligomerization of C16 alpha olefin (AO). Boiling point distribution is shown below in Table A.

TABLE A
Boiling Point Distribution
BOILING POINT DISTRIBUTION

| % OFF | BP(F.) |
|---|---|
| IBP | 818 |
| 1.0% | 824 |
| 2.0% | 830 |
| 3.0% | 836 |
| 4.0% | 843 |
| 5.0% | 881 |
| 6.0% | 911 |
| 7.0% | 930 |
| 8.0% | 947 |
| 9.0% | 957 |
| 10.0% | 964 |
| 11.0% | 968 |
| 12.0% | 972 |
| 13.0% | 975 |
| 14.0% | 978 |
| 15.0% | 981 |
| 16.0% | 983 |
| 17.0% | 985 |
| 18.0% | 987 |
| 19.0% | 989 |
| 20.0% | 990 |
| 21.0% | 992 |
| 22.0% | 993 |
| 23.0% | 994 |
| 24.0% | 995 |
| 25.0% | 996 |
| 26.0% | 997 |
| 27.0% | 997 |
| 28.0% | 998 |
| 29.0% | 999 |
| 30.0% | 999 |
| 31.0% | 1000 |
| 32.0% | 1001 |
| 33.0% | 1001 |
| 34.0% | 1002 |
| 35.0% | 1002 |
| 36.0% | 1003 |
| 37.0% | 1004 |
| 38.0% | 1004 |
| 39.0% | 1005 |
| 40.0% | 1005 |
| 41.0% | 1006 |
| 42.0% | 1006 |
| 43.0% | 1006 |
| 44.0% | 1007 |
| 45.0% | 1007 |
| 46.0% | 1008 |
| 47.0% | 1008 |
| 48.0% | 1009 |
| 49.0% | 1009 |
| 50.0% | 1010 |
| 51.0% | 1010 |
| 52.0% | 1010 |
| 53.0% | 1011 |
| 54.0% | 1011 |
| 55.0% | 1012 |
| 56.0% | 1012 |
| 57.0% | 1012 |
| 58.0% | 1013 |
| 59.0% | 1013 |
| 60.0% | 1014 |
| 61.0% | 1014 |
| 62.0% | 1015 |
| 63.0% | 1016 |
| 64.0% | 1016 |
| 65.0% | 1017 |
| 66.0% | 1017 |
| 67.0% | 1018 |
| 68.0% | 1019 |
| 69.0% | 1019 |
| 70.0% | 1020 |
| 71.0% | 1021 |
| 72.0% | 1021 |
| 73.0% | 1022 |
| 74.0% | 1023 |
| 75.0% | 1023 |
| 76.0% | 1024 |
| 77.0% | 1025 |
| 78.0% | 1026 |
| 79.0% | 1027 |
| 80.0% | 1029 |
| 81.0% | 1031 |
| 82.0% | 1040 |
| 83.0% | 1061 |
| 84.0% | 1076 |
| 85.0% | 1085 |
| 86.0% | 1090 |
| 87.0% | 1095 |
| 88.0% | 1098 |
| 89.0% | 1102 |
| 90.0% | 1105 |
| 91.0% | 1107 |
| 92.0% | 1110 |
| 93.0% | 1114 |
| 94.0% | 1117 |
| 95.0% | 1125 |
| 96.0% | 1153 |
| 97.0% | 1168 |
| 98.0% | 1180 |
| 99.0% | 1209 |
| 100.0% | 1281 |

### Example 6: Oleic Acid Process Feed

A sample of soy-based oleic acid was analyzed by gas chromatography (maximum oven temperature of 380° C.) and also by GC/MS (maximum oven temperature of 355° C.).

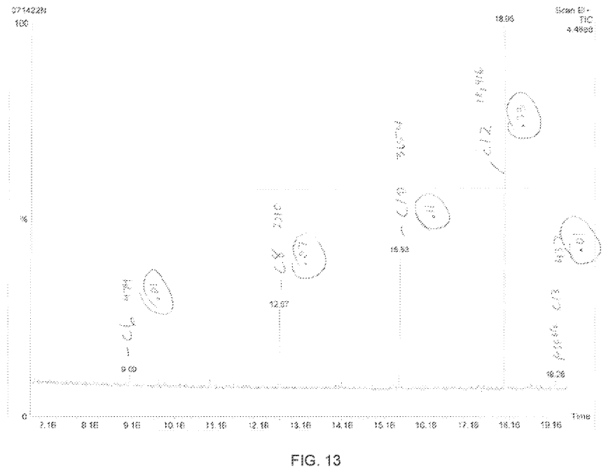

Analysis by gas chromatography showed that very little (0.08%) of the material was in the form of triglycerides with diglyceride content at 1.14%. The remaining material (98.79%) was essentially all fatty acids which are detailed in the GC/MS shown in FIG. 13 and Table B. The bulk of the fatty acids were C18, with C18:1 (oleic) making up approximately 79.19% of the total sample by peak area. No other compounds were readily detected. Several peaks appear to be fatty acids but were not conclusively identified by the instrument (reported as “Other” in Table B). A separate breakout of the individual free fatty acids expressed as a percent of the total free fatty acid component is given in Table C. All the components are expressed as a percentage of the total sample and are determined by peak area. Results should be considered approximate.

TABLE B
Oleic Acid Sample

| COMPONENT | PERCENT OF SAMPLE |
|---|---|
| Triglycerides | 0.08 |
| Diglycerides | 1.14 |
| Glycerol | Not Detected |
| Fatty Acids | 98.79 |
| Monoglycerides | Not Detected |
| Tocopherols | Not Detected |
| Sterols | Not Detected |
| Other | Not Detected |

TABLE C
Total free fatty acid component

| FATTY AO D COMPONENT | PERCENT OF FATTY ACIDS |
|---|---|
| C6:0 | 0.01 |
| C8:0 | 0.07 |
| C10:0 | 0.11 |
| C12:0 | 0.33 |
| C13:0 | 0.01 |
| C14:1 | 0.62 |
| C14:0 | 2.27 |
| C16:1 (cis and trans possible) | 4.27 |
| C16:0 | 3.07 |
| C17:0 | 1.27 |
| C18:3 | Not Detected |
| C18:2 | 4.82 |
| C18:1 (cis and trans possible) | 79.19 |
| C18:0 | 1.62 |
| C20:2 | 0.10 |
| C20:1 (cis and trans possible) | 0.56 |
| C20:0 | 0.07 |
| Unidentified | 1.62 |
| TOTAL: | 100.0 |

### Example 7: Soy Oil Transesterification

A sample of soy oil was converted into methyl esters by transesterification the results were analyzed by gas chromatography (maximum oven temperature of 380° C.) and GC/MS (maximum oven temperature of 355° C.).

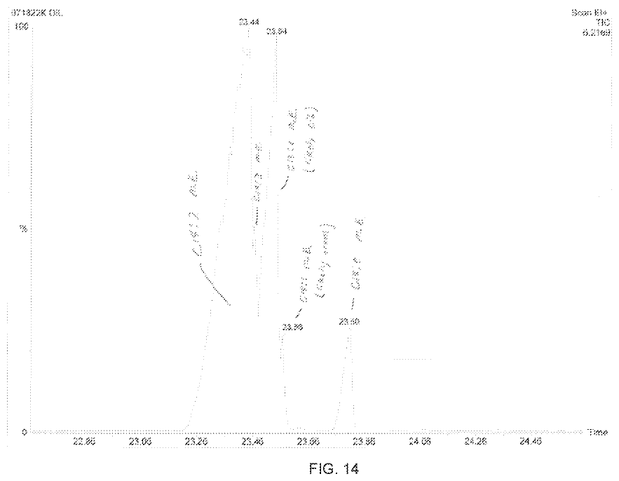

The transesterification was performed using a 6:1 molar ratio of potassium methoxylate to soy oil, with 1% (w/vol) potassium as the catalyst. The reaction was allowed to sit overnight for glycerin separation, followed by centrifugation of the ester layer to further clean up the sample. Analysis by gas chromatography showed that the transesterification was successful with less than 3% di- and triglycerides remaining. The GC/MS sheet shown in FIG. 14 detail the methyl esters and other compounds detected, with the methyl ester composition detailed in Table D. The individual methyl esters are expressed as a percent of the total methyl ester content. The results should be considered approximate.

TABLE D
Methyl Ester Component

| METHYL ESTER COMPONENT | PERCENT OF METHYL ESTERS |
|---|---|
| C14:0 | 0.06 |
| C16:1 | 0.06 |
| C16:0 | 10.81 |
| C17:0 | 0.08 |
| C18:3 | 5.72 |
| C18:2 | 50.74 |
| C18:1 (cis) | 25.06 |
| C18:1 (trans) | 1.87 |
| C18:0 | 4.74 |
| C20:2 | 0.02 |
| C20:1 | 0.15 |
| C20:0 | 0.31 |
| C21:0 | 0.02 |
| C22:0 | 0.26 |
| C23:0 | 0.03 |
| C24:0 | 0.07 |
| TOTAL: | 100.0 |

### REFERENCES

1. Baralt, E., Chen, C. Y., Hao, Y., Liwenny, H. O., Willbe, H. O., Pradhan, A., . . . & Wells, J. (2021). “Process for preparing hydrocarbon mixture exhibiting unique branching structure.” U.S. Pat. No. 10,961,167. Washington, DC: U.S. Patent and Trademark Office.
2. Brennan, J., Mobil Oil Corp, 1973. Dimerization of olefins with boron trifluoride. U.S. Pat. No. 3,742,082.
3. Bullock, C., Yang, N., Cooper, K. and Temme, N., ExxonMobil Chemical Patents Inc, 2006. Blend comprising group III and group IV base stocks. U.S. patent application Ser. No. 11/338,456.
4. Grubbs, R. H., Nguyen, S. T. and Johnson, L. K., California Institute of Technology CalTech, 1999. High activity ruthenium and osmium metal carbene complexes for olefin metathesis reactions. U.S. Pat. No. 5,969,170.
5. Miller, S. J., Chevron Research Co, 1992. Process for producing lube oil from olefins by isomerization over a silicoaluminophosphate catalyst. U.S. Pat. No. 5,082,986.
6. Ohler, N., Fisher, K. and Tirmizi, S., Amyris Inc, 2018. Base oils and methods for making the same. U.S. Pat. No. 9,862,906.
7. Shubkin, R., Ethyl Corp, 1973. Synthetic lubricants by oligomerization and hydrogenation. U.S. Pat. No. 3,780,128.
8. Wang, K., Wu, M. M. S., Galuska, A. A. and Stern, D. L., ExxonMobil Research and Engineering Co, 2014. Process for making basestocks from renewable feedstocks. U.S. Pat. No. 8,772,562.
9. Wu, M. M., Pafford, B. J. and Stavens, K. B., ExxonMobil Chemical Patents Inc, 2014. Polyalphaolefins by Oligomerization and Isomerization. U. S. patent application Ser. No. 14/001,990.
10. Madgavkar, A. M. and Swift, H. E., 1983. Fixed-bed catalytic process to produce synthetic lubricants from decene-1. Industrial & engineering chemistry product research and development, 22 (4), pp. 675-680.
11. J. C. Mol, 20220. “Application of olefin metathesis in oleochemistry: an example of green chemistry”. Green Chemistry, 4, 5-13 (2002);
12. Jackam, J. P., Pierce, J. M., Jones, J. D., 2014. Production of Biodiesel and Glycerin from High Free Fatty Acid Feed Stocks. REG Seneca, LLC. Assignee. Patent US 20140194635 A1. 6 Jan. 2014.
13. Jeske, Kai, et al. ACS catalysis 11.8 (2021): 4784-4798. Noureddini, H., Harkey, D. W. and Gutsman, M. R. (2004), J Amer Oil Chem Soc, 81:203-207.
14. Van der Klis, F., Le Nôtre, J., Blaauw, R., van Haveren, J. and van Es, D. S., 2012. Renewable linear alpha olefins by selective ethenolysis of decarboxylated unsaturated fatty acids. European Journal of Lipid Science and Technology, 114 (8), pp. 911-918.
15. Lacombe, S., Revel, R., Briot, P., Llido, E., Euzen, P., Bobin, C. (2006). Process for Co-producing Olefins and Esters by Ethenolysis of Unsaturated Fats in Non-aqueous Ionis Liquids (US 2006/0079704 A1). U.S. Patent and Trademark Office.
16. Van Der Klis, F., Le Notre, J., Blaauw, R., Van Haveren, J., S Van Es, D., (2012). Renewable linear alpha olefin by selective ethenolysis of decarboxylated unsaturated fatty acids. Eur. J. Lipid Sci. Technol. 2012, 114, 911-918
17. Sondre H. Hopen Eliasson, Anamitra Chatterjee, Giovanni Occhipinti, and Vidar R. Jensen. ACS Sustainable Chemistry & Engineering (2019) 7 (5), 4903-4911. DOI: 10.1021/acssuschemeng.8b05523

What is claimed is:

1. A method for preparing a base oil from a renewable oil comprising triglycerides comprising:
f) acidifying the renewable oil to produce a mixture comprising;
i) free fatty acid mixture comprising;
saturated free fatty acids and unsaturated free fatty acids, and
ii) glycerin;
g) isolating the glycerin from the mixture of fatty acids;
h) separating the saturated free fatty acids from the unsaturated free fatty acids;
i) subjecting the unsaturated free fatty acids to ethenolysis to prepare a mixture comprising;
i) alpha olefins; and
ii) short-chain unsaturated fatty acids, optionally C6-C12 or C8-C12 short chain unsaturated fatty acids; and
j) combining the glycerin from a) with at least a portion of the short chain unsaturated fatty acid of d) to produce a mixture and subjecting the mixture to glycerolysis.

2. The method of claim 1, the method further comprising decarboxylating at least a portion of the short chain unsaturated fatty acids of d) to produce saturated hydrocarbons, optionally wherein the decarboxylation comprises a catalyst or gas phase decarboxylation, optionally a Ni/C catalyst or an oxidative metal catalyst (e.g. silver (II)).

3. The method of claim 1 or 2, wherein in a), the renewable oil is purified prior to acidification.

4. The method of claim 3, wherein purifying the renewable oils comprises clarification, degumming, bleaching, and/or filtering.

5. The method of claim 1, wherein in a), acidifying the renewable oil comprises contacting the renewable oil with an aqueous acid and an organic solvent to provide an organic fraction and an aqueous fraction, wherein the organic fraction comprises the free fatty acids mixture and the aqueous fraction comprises the glycerin.

6. The method of claim 1 or 5, wherein in a), acidifying the renewable oil comprises heating a mixture of the renewable oil and water at a suitable pressure.

7. The method of claim 6, comprising one or more of the following:
i) a ratio of renewable oil to water ranges from about 5:1 to about 1:5, about 4:1 to about 1:4, about 3:1 to about 1:3, or about 2:1 to about 1:2 based on the total weight of the renewable oil and water;
ii) the mixture is heated to a temperature ranging from about 100° C. to about 350° C., about 200° C. to about 300° C., or about 250° C. to about 275° C.; and
iii) the pressure ranges from about 500 psi to about 1000 psi, about 700 psi to about 900 psi, or about 800 psi to about 900 psi.

8. The method of any one of claims 5-7, wherein the acidifying is repeated more than once.

9. The method of any one of claims 5-8, wherein the acid comprises at least one of H₂SO₄, HCl, and H₃PO₄.

10. The method of any one of claims 5-9, wherein the organic fraction comprises 90 wt. % to about 100 wt % free fatty acids and about 0 wt. % to about 10 wt. % glycerin.

11. The method of claim 10, wherein the organic fraction comprises about 90 wt. % free fatty acids and about 10 wt. % glycerine and/or glycerol.

12. The method of claim 11, wherein the organic fraction comprises at least about 50 to about 100 wt. %, about 60 to about 100 wt. %, about 70 to about 100 wt. %, about 80 to about 100 wt. %, about 90 to about 100%, about 60 to about 90 wt. %, or about 70 to about 80 wt. % free fatty acids.

13. The method of claim 12, wherein the separation of the saturated fatty acids from the unsaturated fatty acids in c) comprises temperature dependent solvent extraction.

14. The method of any one of claims 1-13, wherein the saturated free fatty acids of a) are separated into short-chain saturated free fatty acids, optionally C8-12 saturated free fatty acids, and long-chain saturated free fatty acids, optionally C13-C22, C15-C19, or C16-C22 saturated free fatty acids.

15. The method of claim 14, the method further comprising decarboxylation of the long-chain fatty acids.

16. The method of claim 15, wherein the decarboxylation comprises a catalyst selected from Mo on Al₂O₃, MgO on Al₂O₃, and Ni on Al₂O₃, optionally comprising a single-stage continuous process and/or subcritical water.

17. The method of any one of claims 1-16, wherein the ethenolysis comprises a catalyst, optionally selected from tungsten, molybdenum, rhenium and ruthenium.

18. The method of claim 17, wherein the unsaturated free fatty acids comprise and/or consist of long-chain unsaturated free fatty acids.

19. The method of any one of claims 1-18, further comprising separating the alpha olefins from the short-chain unsaturated fatty acids by oligomerization, optionally in the presence of a heterogenous catalyst, optionally providing an alpha olefin dimer, an alpha olefin trimer, an alpha olefin tetramer, and/or an alpha olefin pentamer.

20. The method of claim 19, wherein the heterogenous catalyst is selected from metals, metal oxides, metal salts, or organic materials (e.g. organic hydroperoxides, ion exchangers, and enzymes).

21. The method of any one of claims 1-20, further comprising isomerizing the alpha olefins, optionally in the presence of hydrogen or under inert conditions.

22. The method of claim 21, wherein isomerization is performed inside a Parr reactor.

23. The method of claim 21 or 22, wherein the temperature condition of isomerization reaction ranges from about 100° C. to about 500° C., about 100° C. to about 200° C., about 200° C. to about 300° C., about 300° C. to about 400° C., or about 400° C. to about 500° C.

24. The method of any one of claims 21-23, wherein the pressure condition of isomerization reaction ranges from about 1,000 psi to about 3,000 psi, from about 1,000 psi to about 2,000 psi, from about 2,000 psi to about 3,000 psi, or from about 1,500 psi to about 2,500 psi.

25. The method of claim 19, wherein the heterogenous catalyst is selected from AlCl₃ and BF₃.

26. The method of any one of claims 1-25, wherein the glycerolysis produces short chain unsaturated acyl-glycerides

27. The method of claim 26, wherein the glycerolysis is base catalyzed, optionally wherein the catalyst a catalyst, optionally wherein the catalyst is a methoxide selected from sodium methoxide, potassium methoxide, lithium methoxide, zinc methoxide, calcium methoxide, tributyltin methoxide, magnesium methoxide, tantalum (V) methoxide, titanium (IV) methoxide, antimony (III) methoxide, germanium methoxide, copper (II) methoxide, and combinations thereof.

28. A method for preparing a base oil from a renewable oil comprising triglycerides comprising:
e) transesterifying the renewable oil to produce a mixture comprising;
i) fatty acid ester mixture comprising;
saturated fatty acid esters and unsaturated fatty acid esters, and
ii) glycerin;
f) isolating the glycerin from the fatty acid ester mixture;
g) separating the saturated fatty acid esters from the unsaturated fatty acid esters;
h) subjecting the unsaturated fatty acid esters to ethenolysis to prepare a mixture comprising;
i) alpha olefins; and
ii) short-chain unsaturated fatty acid esters, optionally C6-C12 or C8-C12 short chain unsaturated fatty acid esters.

29. The method of claim 28, wherein in a), the renewable oil is purified prior to acidification.

30. The method of claim 29, wherein purifying the renewable oils comprises clarification, degumming, bleaching, and/or filtering.

31. The method of claim any one of claims 1-30, wherein in a), transesterifying comprises reacting the renewable oil with an alcohol, optionally methanol, optionally in the presence of a catalyst.

32. The method of claim 12, wherein the separation of the saturated fatty acid esters from the unsaturated fatty acid esters in c) comprises temperature dependent solvent extraction.

33. The method of any one of claims 1-13, wherein the saturated fatty acid esters of a) are separated into short-chain saturated fatty acid esters, optionally C8-12 saturated fatty acid esters, and long-chain saturated fatty acid esters, optionally C13-C22, C15-C19, or C16-C22 saturated fatty acid esters.

34. The method of claim 28, further comprising:
i) converting at least a portion of the short chain unsaturated fatty acid esters of d) into short chain unsaturated fatty acids; and
j) decarboxylating the short chain unsaturated fatty acids of e) to produce saturated hydrocarbons, optionally wherein the decarboxylation comprises a catalyst or gas phase decarboxylation, optionally a Ni/C catalyst or an oxidative metal catalyst (e.g. silver (II)).

35. The method of claim 33, the method further comprising:
k) converting the long-chain fatty acid esters into long chain fatty acids; and
l) decarboxylation of at least a portion of the long-chain fatty acids of g) to produce saturated hydrocarbons, optionally wherein the decarboxylation comprises a catalyst or gas phase decarboxylation, optionally a Ni/C catalyst or an oxidative metal catalyst (e.g. silver (II)).

36. The method of claim 34 or 35, wherein the decarboxylation comprises a catalyst selected from Mo on Al₂O₃, MgO on Al₂O₃, and Ni on Al₂O₃, optionally comprising a single-stage continuous process and/or subcritical water.

37. The method of any one of claims 28-36, wherein the ethenolysis comprises a catalyst, optionally selected from tungsten, molybdenum, rhenium and ruthenium.

38. The method of claim 37, wherein the unsaturated fatty acid esters comprise and/or consist of long-chain unsaturated fatty acid esters.

39. The method of any one of claims 28-38, further comprising separating the alpha olefins from the short-chain unsaturated fatty acid esters by oligomerization, optionally in the presence of a heterogenous catalyst, optionally providing an alpha olefin dimer, an alpha olefin trimer, an alpha olefin tetramer, and/or an alpha olefin pentamer.

40. The method of claim 39, wherein the heterogenous catalyst is selected from metals, metal oxides, metal salts, or organic materials (e.g. organic hydroperoxides, ion exchangers, and enzymes).

41. The method of any one of claims 28-40, further comprising isomerizing the alpha olefins, optionally in the presence of hydrogen or under inert conditions.

42. The method of claim 41, wherein isomerization is performed inside a Parr reactor.

43. The method of claim 41 or 42, wherein the temperature condition of isomerization reaction ranges from about 100° C. to about 500° C., about 100° C. to about 200° C., about 200° C. to about 300° C., about 300° C. to about 400° C., or about 400° C. to about 500° C.

44. The method of any one of claims 41-43, wherein the pressure condition of isomerization reaction ranges from about 1,000 psi to about 3,000 psi, from about 1,000 psi to about 2,000 psi, from about 2,000 psi to about 3,000 psi, or from about 1,500 psi to about 2,500 psi.

45. The method of claim 44, wherein the heterogenous catalyst is selected from AlCl₃ and BF₃.

46. The method of claim 34, wherein the method further comprises combining the glycerin from a) with at least a portion of the short chain unsaturated fatty acids of e) to produce a mixture and subjecting the mixture to glycerolysis.

47. The method of claim 46, wherein the glycerolysis produces short chain unsaturated acyl-glycerides

48. The method of claim 46 or 47, wherein the glycerolysis is base catalyzed, optionally wherein the catalyst a catalyst, optionally wherein the catalyst is a methoxide selected from sodium methoxide, potassium methoxide, lithium methoxide, zinc methoxide, calcium methoxide, tributyltin methoxide, magnesium methoxide, tantalum (V) methoxide, titanium (IV) methoxide, antimony (III) methoxide, germanium methoxide, copper (II) methoxide, and combinations thereof.

49. The method of any one of claims 1-48, wherein the renewable oil comprises or consists of one or more selected from seed oil, vegetable oil, and animal derived oils.

50. The method of claim 49, wherein the renewable oil is selected from rapeseed oil, soy oil, castor oil.

51. The method of claim 49, wherein the renewable oil is derived from one or more of poultry, beef, and fish.

52. A base oil prepared from the method of any one of claims 1-51.

53. Renewable alpha olefins prepared from the method of any one of claims 1-51.

54. Renewable diesel prepared from the method of any one of claims 1-51.

55. Synthetic gasoline prepared from the method of any one of claims 1-51.

56. Unsaturated acyl glycerides prepared from the method of any one of claims 1-51.

57. Unsaturated acyl glycerides prepared from the method of any one of claims 1-51.

58. A lubricant comprising:
a) a saturated hydrocarbon base oil in an amount ranging from about 50 wt % to about 70 wt % of the total weight of the lubricant, wherein the saturated hydrocarbon base oil comprises oligomers of C14-C18 olefin monomers, the oligomers having an average carbon number in a range of from 29 to 36;
b) a viscosity modifier in an amount ranging from about 1 wt % to about 30 wt %, optionally about 1.4 wt %, about 1.80 wt %, about 3.2 wt %, about 4.13 wt %, about 5.2 wt %, about 16.25 wt %, or about 26 wt %, of the total weight of the lubricant;
c) a detergent in an amount ranging from about 10 wt % to about 15 wt %, optionally about 12.3 wt %, of the total weight of the lubricant; and
d) a pour point depressant in an amount ranging from about 0.1 wt % to about 1 wt %, optionally about 0.3 wt %, of the total weight of the lubricant.

59. The lubricant of claim 58, wherein the oligomers comprise and/or consist of dimers, trimers, tetramers, and/or pentamers, optionally dimers.

60. The lubricant of claim 58, wherein the saturated hydrocarbon base oil exhibits one or more of the following properties:
a) a Noack Volatility as measured by ASTM D5800 and/or CEC L-40-A-93 that is less than about 14%, less than about 13%, less than about 12%, less than about 11%, less than about 10%, or less than about 9%, optionally about 7.4%;
b) a Bromine Index below about 1000 mg Br2/100 g, about 500 mg Br2/100 g, or below about 200 mg Br2/100 g as determined in accordance with D2710-09;
c) an average branching index (BI) as determined by 1H NMR that is in the range of about 22 to about 26;
d) an average paraffin branching proximity (BP) as determined by 13C NMR in a range of from about 18 to about 26;
e) a viscosity index as determined in accordance with ASTM D2270 of about 125 or greater, about 130 or greater, about 135 or greater, or about 140 or greater;
f) a pour point as determined in accordance with ASTM D97 less than about −20° C., less than about −27° C., less than about −30° C., less than about −33° C., less than about −36° C., less than about −39° C., or less than about-42° C.;
g) a Cold Crank Simulated (CCS) dynamic viscosity as measured by ASTM D5293 at −35° C. less than about 1800 cP, less than about 1700 cP, less than about 1600 cP, less than about 1500 cP, less than about 1400 cP, less than about 1300 cP, less than about 1200 cP, or less than about 1100 cP; and
h) a KV (100) as measured by ASTM D445-17a that is in the range of about 3.7 cSt to about 9.7 cSt, or about 3.7 cSt to about 4.8 cSt.

61. The lubricant of claim 58-60, wherein the saturated hydrocarbon base oil comprises SynNova 4 in an amount ranging from about 50 wt % to about 60 wt % of the total weight of the lubricant, and SynNova 9 in an amount ranging from about 3 wt % to about 7 wt % of the total weight of the lubricant.

62. The lubricant of any one of claims 58-61, wherein the viscosity modifier comprises one or more of Infineum SV603 and Infineum SV261L, the detergent comprises Infineum P6003, and the pour point depressant comprises Infineum V385.

63. The lubricant of any one of claims 58-62, wherein the lubricant comprises:
a) a saturated hydrocarbon base oil comprising SynNova 4 in an amount ranging from about 56 wt % to about 57 wt % of the total weight of the lubricant, and SynNova 9 in an amount ranging from about 4.5 wt % to about 5.5 wt % of the total weight of the lubricant;
b) a viscosity modifier comprising Infineum SV603 in an amount ranging from about 25.5 wt % to about 26.5 wt % of the total weight of the lubricant;
c) a detergent comprising Infineum P6003 in an amount ranging from about 12 wt % to about 13 wt % of the total weight of the lubricant; and
d) a pour point depressant comprising Infineum V385 in an amount ranging from about 0.2 wt % to about 0.4 wt % of the total weight of the lubricant.
